(19) United States
(12) Patent Application Publication
(10) Pub. No.: US 2009/0280166 A1
(43) Pub. Date: Nov. 12, 2009

(54) FERRITIN AS A THERAPEUTIC TARGET IN ABNORMAL CELLS

(75) Inventors: James R. Connor, Hershey, PA (US); Nodar Surguladze, Hershey, PA (US)

Correspondence Address: J. Rodman Steele;Novak Druce & Quigg LLP, 525 Okeechobee Blvd, Suite 1500, West Palm Beach, FL, 33401, US

(73) Assignee: The Penn State Research Foundation, University Park, PA (US)

(21) Appl. No.: 12/422,709

(22) Filed: Apr. 13, 2009

Related U.S. Application Data
(63) Continuation of application No. 11/457,667, filed on Jul. 14, 2006.
(60) Provisional application No. 60/699,554, filed on Jul. 15, 2005.
(60) Provisional application No. 60/728,140, filed on Oct. 19, 2005.

(51) Int. Cl. A61K 9/127; A61P 35/00; A61K 31/7105

(52) U.S. Cl. 424/450; 514/44 A

(57) ABSTRACT

Compositions for treatment of iron related diseases comprise an inhibitor of ferritin. An inhibitor of ferritin is active to reduce the level of H ferritin protein in a cell and/or to reduce the activity of H ferritin in a cell. Compositions providing cytoprotection, regulation of iron, increasing longevity and viability of cells are described.

### CROSS REFERENCE TO RELATED APPLICATIONS

The present application is a continuation of prior application Ser. No. 11/457,667 filed Jul. 14, 2006, which claims the priority of U.S. provisional patent application No. 60/699,554, entitled “NUCLEAR FERRITIN IN TUMOR CELLS,” filed Jul. 15, 2005; and U.S. provisional patent application No. 60/728,140, entitled “FERRITIN AS THERAPEUTIC TARGET IN TUMOR CELLS,” filed Oct. 19, 2005. The present application claims the benefit of the foregoing applications which are incorporated herein by reference in their entirety.

### FIELD OF THE INVENTION

The invention provides compositions and methods for highly selective targeting of H-ferritin. The compositions comprise siRNA's which bind in a sequence dependent manner to their target genes and inhibit expression of undesired nucleic acid sequences in a target cell. When administered into cells, siRNA's cause elimination or degradation of a non-essential extra-chromosomal genetic element. Inhibitor compositions of H-ferritin are provided.

### BACKGROUND

Ferritin is a large multi-subunit iron storage protein with 24 polypeptide subunits having a molecular weight of nearly 480,000 Da. This multi-subunit protein is capable of containing as many as 4,500 atoms of iron within a hydrous ferric oxide core. Mammalian ferritin contains two distinct subunit classes, H and L, which share about 54% identity. The H and L subunits appear to have different functions: the L subunit enhances the stability of the iron core while the H subunit has a ferroxidase activity that appears to be necessary for the rapid uptake of ferrous iron. H subunit rich ferritins are localized in tissues undergoing rapid changes in local ion concentration. For instance, expression of the H subunit is preferentially increased relative to the L subunit in cells undergoing differentiation development proliferation and metabolic stress.

A need in the art exists for development of drugs that are therapeutically effective against tumors and other iron related disorders.

### SUMMARY OF THE INVENTION

Sequence specific siRNA bind to a target nucleic acid molecule, inhibiting the expression thereof. siRNA's are effective in the treatment of abnormal cells, abnormal cell growth and tumors, including those tumors caused by infectious disease agents, and iron related disorders. Compositions for delivery of siRNA and methods of treatment thereof are provided.

It is now found that the H subunit of ferritin may play a protective role, for instance protecting cells (“cytoprotective” effect”) from the oxidative effects of iron. Iron can produce highly reactive free radicals which can damage cells. In humans, oxidative cell and tissue damage has been linked to carcinogenesis, liver cirrhosis, fibrosis hepatitis, neurodegenerative disorders, autoimmune diseases, and atherosclerosis, among others. While all forms of life require significant quantities of iron for survival and reproduction, its localization and levels must be carefully regulated in order to avoid oxidative damage that can produce consequences such as cell degeneration and consequent disease.

In a preferred embodiment, a composition is provided according to the present invention which includes an inhibitor of ferritin. In a preferred embodiment, a composition according to the present invention includes an inhibitor of H ferritin. An inhibitor of H ferritin is active to reduce the level of H ferritin protein in a cell and/or to reduce the activity of H ferritin in a cell. An inhibitor of H ferritin active to reduce the level of H ferritin protein in the cell may be an inhibitor of transcription and/or translation of H ferritin. In addition, an inhibitor of H ferritin active to reduce the level of H ferritin protein in the cell may stimulate degradation of the H ferritin protein and/or H ferritin encoding RNA. An inhibitor of ferritin transcription and/or translation may be a nucleic acid-based inhibitor such as an antisense oligonucleotides complementary to a target H ferritin mRNA, as well as ribozymes and DNA enzyme which are catalytically active to cleave the target mRNA.

A method of treating cancer in an individual having a tumor is provided which includes administration of a composition according to the present invention. Methods of treatment of an individual having a tumor optionally further include administration of an anti-tumor treatment are provided. Exemplary anti-tumor treatments include radiation administration including external radiation therapy and/or internal administration of radiation such as by implant radiation. Administration of a composition according to the invention along with an anti-tumor treatment is advantageous over administration of an anti-tumor treatment alone since a synergistic effect of the combined treatments may be seen. Thus, the dose of an administered anti-tumor treatment is lower than would otherwise be required for an anti-tumor effect.

In one embodiment, an inhibitor of H ferritin is small interfering RNA against H ferritin.

In a preferred embodiment a method of inhibiting a tumor cell, comprises administering a composition including an inhibitor of a cytoprotective effect of ferritin in a tumor cell. Preferably, the composition comprises comprising an inhibitor of an H ferritin protein.

In a preferred embodiment, an inhibitor of H-Ferritin is an inhibitor of nuclear transport of the H ferritin protein.

In another preferred embodiment, an inhibitor of H-Ferritin is an inhibitor of O-glycosylation of an H ferritin protein.

In another preferred embodiment, an inhibitor of H-Ferritin is an inhibitor of synthesis of an H ferritin protein.

In another preferred embodiment, an inhibitor of H-Ferritin is an inhibitor of transcription of an H ferritin protein.

In another preferred embodiment, an inhibitor of H-Ferritin is an inhibitor of a post-translational modification of an H ferritin protein.

In another preferred embodiment, an inhibitor of H-Ferritin is an inhibitor of a cytoprotective effect of H ferritin.

In another preferred embodiment, an inhibitor of H-Ferritin reduces an amount of H ferritin present in a tumor cell, and/or the inhibitor inhibits translocation of H ferritin from tumor cell cytoplasm to a tumor cell nucleus, and/or the inhibitor inhibits transcription of H ferritin in a tumor cell, and/or the inhibitor inhibits translation of H ferritin in a tumor cell.

In another preferred embodiment, an inhibitor of H-Ferritin comprises an antisense nucleic acid capable of specifically binding to at least a portion of an H ferritin nucleic acid and inhibiting transcription and/or translation of the H ferritin nucleic acid. Preferably, the inhibitor comprises a small interfering RNA comprising at least one of SEQ ID NO's: 1-8.

In a preferred embodiment, combinations of siRNAs comprising any one of SEQ ID NO's: 1-8 are used to treat a patient suffering from cancer or other iron related diseases such as for example, fibrosis hepatitis, neurodegenerative disorders, autoimmune diseases, and atherosclerosis, among others.

In another preferred embodiment, the composition further comprises a pharmaceutically acceptable carrier.

In another preferred embodiment, the composition comprises a particulate delivery vehicle, the vehicle comprising a tumor cell targeting moiety such as an antibody, nucleic acid, and/or receptor ligand, the vehicle associated with the inhibitor. Preferably, the particulate delivery vehicle is capable of intracellular delivery of the inhibitor, such as, for example, a liposome.

In another preferred embodiment, the composition comprises an inhibitor of O-glycosylation. An example of an inhibitor of O-glycosylation is alloxan.

In another preferred embodiment, the method of treating a cancer patient further comprises the step of administering an anti-tumor agent and/or an anti-tumor treatment. Preferably, the anti-tumor agent is associated with a particulate delivery vehicle and the anti-tumor treatment is a radiation treatment, surgery and/or chemotherapy.

In another preferred embodiment, a pharmaceutical composition comprises an inhibitor of a ferritin protein wherein the ferritin protein is an H ferritin protein.

In another preferred embodiment, a pharmaceutical composition comprises an inhibitor of H-Ferritin which reduces an amount of H ferritin present in a tumor cell, and/or the inhibitor inhibits translocation of H ferritin from tumor cell cytoplasm to a tumor cell nucleus, and/or the inhibitor inhibits transcription of H ferritin in a tumor cell, and/or the inhibitor inhibits translation of H ferritin in a tumor cell.

In another preferred embodiment, the pharmaceutical composition comprises an inhibitor of H-Ferritin comprising an antisense nucleic acid capable of specifically binding to at least a portion of an H ferritin nucleic acid and inhibiting transcription and/or translation of the H ferritin nucleic acid., and/or a chemotherapeutic agent. Preferably, the inhibitor comprises a small interfering RNA comprising at least one of SEQ ID NO's: 1-8.

In another preferred embodiment, the inhibitor is associated with a particulate delivery vehicle. Preferably, the particulate delivery vehicle is a liposome. Preferably, a chemotherapeutic agent is associated with a particulate delivery vehicle.

In another preferred embodiment, the particulate delivery vehicle further comprises a targeting moiety for targeting a specified cell type. For example, a targeting moiety is an antibody specific for a tumor antigen, nucleic acid, and/or receptor ligand.

A pharmaceutical composition comprising a particulate delivery vehicle associated with an inhibitor of H ferritin. Preferably, the pharmaceutical composition further comprises a particulate delivery vehicle associated with an anti-tumor agent. Preferably, the particulate delivery vehicle further comprises a targeting moiety for targeting a specified cell type. For example, a tumor cell targeting moiety is as an antibody, nucleic acid, and/or receptor ligand.

In a preferred embodiment, the particulate delivery vehicle is a liposome.

In another preferred embodiment, a method of treating iron-related disorders comprises administering to a patient a composition comprising an inhibitor of ferritin to treat a patient suffering from iron-related diseases. These disease are characterized by an iron-imbalance, i.e. excess iron or iron-deficiency.

In another preferred embodiment, a patient suffering from iron-deficiency related disorders is treated with a composition comprising H-ferritin and/or inducers of H-ferritin. Treatment, using the compositions of the invention include administration of H-ferritin, e.g. SEQ ID NO: 9 and/or NLS-ferritin, in a pharmaceutical composition and/or delivery vehicle such as a liposome which comprises a targeting moiety such as antibody, receptor, ligand etc. Also within the scope of the invention are use of vectors expressing H-ferritin, e.g. SEQ ID NO: 9 and/or NLS-ferritin under the control of a tissue specific promoter or inducible promoter. The administration of H-ferritin can be combined with one or more other treatments such as EPO (erythropoietin) to stimulate bone marrow.

In another preferred embodiment, a method of increasing the viability and/or longevity of a cell comprises administering compositions of H-ferritin, e.g. SEQ ID NO: 9 and/or NLS-ferritin, and/or delivery vehicle such as a liposome which comprises a targeting moiety such as antibody, receptor, ligand etc. Also within the scope of the invention are use of vectors expressing H-ferritin, e.g. SEQ ID NO: 9 and/or NLS-ferritin under the control of a tissue specific promoter or inducible promoter. Such compositions are useful in long term cell cultures, such as for example, ex-vivo growth of cells for re-implantation in a patient in need of such therapy, such as transplants, bone-marrow transplants and the like.

Other aspects are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a graph showing the effects of siRNA against H ferritin in combination with Temodar (triangles) on U251 compared to control RNA sequences in combination with Temodar (rectangles) on the same cells.

FIG. 2 is a graph showing the effects of siRNA against H ferritin in combination with Temodar (triangles) on SW1088 cells, compared to control RNA sequences in combination with Temodar (rectangles) on the same cells.

FIG. 3 is a graph showing the effects of siRNA against H ferritin in combination with BCNU (triangles) on U251 cells, compared to control RNA sequences in combination with BCNU (rectangles) on the same cells.

FIG. 4 is a graph showing the effects of siRNA against H ferritin in combination with BCNU (triangles) on SW1088 cells, compared to control RNA sequences in combination with BCNU (rectangles) on the same cells.

FIG. 5 is a scan of a confocal image of SW1088 cells. Human grade III astrocytoma cells (SW1088) were fixed, and incubated with polyclonal rabbit anti-human H-ferritin antibody at 1:200 dilution, followed by Alexa 488-conjugated goat anti-rabbit IgG at 1:200 dilution. Nuclei were visualized by DAPI staining at a final concentration of 100 ng/ml. Alexa- and DAPI-fluorescence emissions (shown in green and blue respectively) were observed using a confocal microscope with illumination at 488 nm (Alexa) and 360 nm (DAPI).

FIGS. 6A and 6B are graphs showing the distribution of ferritin in subnuclear fractions and oligomerization of nuclear ferritin. FIG. 6A shows total nuclear extract and different fractions (soluble nuclear fraction, nuclease-digested fractions, nuclear matrix and nucleoli) were prepared as described. Samples containing 20 μg of protein were resolved by SDS/PAGE and ferritin contents were detected by Western blotting using the HS-59 mouse anti-rH-ferritin antibody as a probe. Immunocomplexes were detected using peroxidase-conjugated goat anti-mouse IgG. Images were captured on a film and relative band intensities were estimated by densitometry. The results are presented as band intensities normalized to that of the unfractionated nuclear sample. The error bars represent S.D. values for triplicate samples obtained from independent cell preparations. FIG. 7B shows total nuclear extract (20 μg of protein/sample) resolved by SDS/PAGE. Ferritin was detected by Western blotting using HS-59 mouse anti-rH-ferritin antibody as described. The intensities of bands with mobilities corresponding to the monomeric subunit of ferritin (Mᵣ 21 094) and subunit dimers, subunit trimers and higher subunit oligomers are represented as a percentage of the summed band intensities. Increasing the concentrations of SDS and 2-mercaptoethanol (up to 4% and 7 mM respectively) as well as increasing the sample boiling time did not change the ratio of different multimers. The inset shows a representative gel lane with bands designated M (subunit monomer), D (subunit dimer) and T (subunit trimer). An additional, faint band with mobility intermediate to that of ferritin subunit monomer and subunit dimer is regularly seen. The error bars represent S.D. values for three independent samples.

FIGS. 7A and 7B are graphs showing that nuclear and cytoplasmic H-ferritins are translated from the same mRNA. SW1088 cells were transfected with anti-H-ferritin siRNA. The cells were transfected and then plated in flasks (Western-blot analysis) or on coverslips (for immunohistochemical analysis). The data from Western-blot analysis are shown in FIG. 7A. Cells for biochemical analysis were suspended, lysed and the relative ferritin contents of whole cell extracts were determined at the indicated times by Western blotting. Results are expressed as band intensities normalized to the ferritin content of the parent, untransfected SW1088 cells, sampled at the time of transfection. ▪, cells transfected with siRNA against human H-ferritin; ▴, cells transfected with non-specific RNA; ♦, cells exposed to mock transfection using a buffer instead of RNA solution. For the immunohistochemical analysis (FIG. 7B), the cells were fixed and immunostained for H-ferritin as described at the indicated times. For each time period, three different slips were examined and, within each slip, multiple (≧3) microscopic fields were captured for analysis. Nuclear ferritin content was analyzed on the basis of the fluorescence intensities of entire nuclear regions. The results are presented normalized to a control value obtained with nuclei subjected to mock transfection using buffer instead of siRNA. The transfection efficiency was determined to be ≧90% using rhodamine-conjugated non-specific RNA. In both FIGS. 7A and 7B, the error bars represent S.D. values. The similar pattern of decrease in nuclear and whole-cell H-ferritin contents after transfection with siRNA indicates that nuclear and cytoplasmic H-ferritins are expressed from the same message.

FIG. 8 is a scan of a Western blot showing H-ferritin can be immunoprecipitated with a monoclonal antibody raised against GlcNAc. Astrocytoma (SW1088) cells were lysed. Cytoplasmic and nuclear fractions were isolated and 1 mg of total protein from each fraction was pretreated with Protein A/G to precipitate proteins with IgG-like folds. Supernatants were then treated with a monoclonal antibody raised against GlcNAc, and immunocomplexes were precipitated with additional Protein A/G. Precipitated immunocomplexes were subjected to SDS/PAGE and the blots were stained with anti-human H-ferritin polyclonal antibody. Lane a, proteins precipitated from total nuclear extract with an antibody raised against O-GleNac; lane b, nuclear extract proteins remaining in the supernatant after immunoprecipitation; lane c, proteins precipitated from cytoplasmic extract with an antibody raised against O-GlcNac; lane d, cytoplasmic proteins remaining in the supernatant after immunoprecipitation; lane e, total nuclear extract without immunoprecipitation (20 μg of protein was loaded for this sample); lane f, precipitate of anti-O-GlcNac antibody with Protein A/G (no cellular proteins). These results show that O-glycosylated ferritin is found in both the nucleus and cytoplasm. On the basis of densitometric analysis of the band intensity, the ratio of cytoplasmic to nuclear O-glycosylated ferritin is approx. 1.8:1. However, the total amount of ferritin in the cytoplasm is four to six times higher than that found in the nucleus.

FIGS. 9A-9C show nuclear import of ferritin is inhibited by alloxan, whereas cytoplasmic levels of ferritin are not affected. Under resting conditions, SW1088 cells contain ferritin in both nuclear and cytoplasmic compartments. Treatment of cells with the iron chelator DFO significantly decreases ferritin content in both compartments. The reappearance of ferritin in cytoplasmic and nuclear compartments after DFO treatment (alone) or treatment with DFO+alloxan is affected by the presence of alloxan (alx) and/or FAC in the culture medium. FIG. 9A is a schematic illustration of the experimental procedure, showing the time course of changes in culture conditions. FIG. 9B is a scan of Western blots of nuclear (N) and cytoplasmic (C) extracts of SW1088 cells, resolved by SDS/PAGE. Samples of the nuclear extract contained 20 μg of total protein, whereas samples of the cytoplasmic extract contained 10 μM of total protein. Ferritin was detected with HS-59 mouse monoclonal antibody and horseradish peroxidase-conjugated goat anti-mouse IgG. Lane a, extracts from cells cultured in medium containing 100 μM DFO; lane b, extracts of cells cultured in normal medium; lane c, extracts of cells cultured in 100 μM FAC; lane d, extracts of cells cultured in 100 μM DFO+1 mM alloxan; lane e, extracts of cells cultured in normal medium supplemented with 1 mM alloxan; lane f, extracts of cells cultured in medium supplemented with 100 μM FAC+1 mM alloxan. FIG. 9C is a graph showing a summary of ferritin contents. The relative amounts of ferritin in the nuclear (black bars) and cytoplasmic (striped bars) fractions were measured after an initial treatment with DFO alone or DFO+100 μM alloxan and a subsequent culture in the presence of DFO alone, normal medium, medium supplemented with FAC, medium supplemented with DFO+alloxan, normal medium+alloxan or normal medium+FAC and alloxan. The relative amounts of ferritin are normalized to the amount of ferritin in the corresponding fractions before the initial DFO treatment.

FIG. 10 is a graph showing alloxan inhibits protein O-glycosylation in SW1088 human astrocytoma cells. Cells were grown in triplicate independent cultures in the presence of 0, 100, 500 μM and 1 mM concentrations of alloxan. Aliquots of whole cell lysates from each culture were applied to a nitrocellulose membrane using a vacuum slot blot device. The membrane was blocked in a 5% solution of non-fat dry milk at 21±1° C. for 1 h and incubated overnight with mouse monoclonal anti-O-GlcNAc antibody. Immunocomplexes were detected and quantified. As an internal control for this assay, membrane loadings of 1 (♦), 5 (▪) and 10 (▴) μg of total protein were tested and they gave similar responses to changes in the concentration of alloxan in the original cultures. The results show that less O-glycosylated protein is available for detection as [alloxan] increases; parallel testing for cell viability with MTT (inset) shows that, at 1 mM alloxan, 87±3% of the cells remain viable.

FIGS. 11A and 11B show treatment with alloxan does not cause iron-release from ferritin in vitro. Supercoil-relaxation assays were performed with pUC19 plasmid DNA (0.5 μg/assay). FIG. 11A is a scan showing electrophoretic profiles of pUC19 DNA incubated in the presence of recombinant H-ferritin (lanes a-e), recombinant H-ferritin+1 mM alloxan (lanes f-j) and 1 mM alloxan alone (lanes k-o). Reaction times were as indicated. Lane p, a sample of the superhelical pUC19 DNA that was not treated with ferritin. Band assignments: R, relaxed circle form; SC, superhelical topoisomers. FIG. 11B is a graph showing the mole fraction of superhelical DNA as a function of the reaction time for samples containing rH-ferritin (♦), rH-ferritin+1 mM alloxan (▪) and 1 mM alloxan alone (▴). Data were obtained from three experiments similar to that shown in FIG. 11A. Error bars represent S.D. values. Similar rates of DNA relaxation by ferritin both in the presence and absence of alloxan indicate that alloxan does not cause a significant release of iron from ferritin under these conditions.

FIG. 12 shows the H- (SEQ ID NO: 9) and L-ferritin (SEQ ID NO: 10) sequence alignment. The high potential O-glycosylation sites in H-ferritin (shown in boldface and underlined) are found in the N-terminal sequence in a location that does not overlap the L-ferritin sequence. Low probability sites in H-ferritin (shown in boldface, underlined and italicized) also are not found in overlapping regions of the L-ferritin sequence at the C-terminal end.

FIG. 13 is a graph showing the in vivo efficacy of siRNA H-ferritin in a subcutaneous tumor model. The siRNA for H-ferritin or the nonsense (NS) control was first conjugated into liposomes and then injected directly into a subcutaneous glioblastoma tumor growing in the flank of nude mice. The concentration of siRNA or NS RNA injected into the tumor was ˜4 μg. After injection of the siRNA, the mice, received 25 μM of BCNU delivered i.p. 24 hours. The injections were performed once a week.

FIG. 14 is a graph showing growth rate of transfected primary astrocytes. The experimental group consisted of transfection with a Nuclear localization signal on H-ferritin.

FIG. 15 is a graph showing the results of the longevity of transfected astrocytes versus control. Cells were transfected and then plated at equal density.

FIG. 16 is a graph showing the cytotoxicity profile of stress factors on rat primarily astrocytes transfected with NLS and non-NLS H ferritin construct. Cytotoxicity was determined by MTT assay after treatment with three different concentrations of soluble iron compound 3,5,5-trimethyl (hexanoyl) ferrocene (TMHF). A total of 12 trials were run in triplicate for each sample and the grand mean and SE are reported.

### DETAILED DESCRIPTION

Compositions targeting H-ferritin and inhibitors thereof, are described. Methods of treating cancer and iron related disorders using the compositions of the invention are provided. Compositions are also provided that protect cells from stressors, increase cell viability and longevity.

### DEFINITIONS

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

As used herein, the term “ribozymes” refers to linear oligonucleotides with a loop structure, are catalytic nucleic acids, and are designed to inactivate specific mRNA.

As used herein, the term “DNA repair gene” refers to a gene that is part of a DNA repair pathway, that when altered, permits mutations to occur in the DNA of the organism.

As used herein, the terms “exon” and “intron” are art-understood terms referring to various portions of genomic gene sequences. “Exons” are those portions of a genomic gene sequence that encode protein. “Introns” are sequences of nucleotides found between exons in genomic gene sequences. The siRNA's can be targeted to exons and/or to introns.

As used herein, the singular forms “a”, “an” and “the” include plural referents unless the context clearly dictates otherwise.

As used herein, the term “infectious agent” refers to an organism wherein growth/multiplication leads to pathogenic events in humans or animals. Examples of such agents are: bacteria, fungi, protozoa and viruses.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the terms “oligonucleotide”, “siRNA” “siRNA oligonucleotide” and “siRNA's” are used interchangeably throughout the specification and include linear or circular oligomers of natural and/or modified monomers or linkages, including deoxyribonucleosides, ribonucleosides, substituted and alpha-anomeric forms thereof, peptide nucleic acids (PNA), ed nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoögsteen or reverse Hoögsteen types of base pairing, or the like.

The oligonucleotide may be “chimeric”, that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleosides and/or oligonucleotide analogs as described above.

The oligonucleotide can be composed of regions that can be linked in “register”, that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139 (1999); Freier S. M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000),); 2′-O, 3′-C-linked [3.2.0] bicycloarabinonucleosides (see e.g. N. K Christiensen., et al, J. Am. Chem. Soc., 120: 5458-5463 (1998). Such analogs include synthetic nucleosides designed to enhance binding properties, e.g., duplex or triplex stability, specificity, or the like.

The term “stability” in reference to duplex or triplex formation generally designates how tightly an antisense oligonucleotide binds to its intended target sequence; more particularly, “stability” designates the free energy of formation of the duplex or triplex under physiological conditions. Melting temperature under a standard set of conditions, e.g., as described below, is a convenient measure of duplex and/or triplex stability. Preferably, oligonucleotides of the invention are selected that have melting temperatures of at least 45° C. when measured in 100 mM NaCl, 0.1 mM EDTA and 10 mM phosphate buffer aqueous solution, pH 7.0 at a strand concentration of both the oligonucleotide and the target nucleic acid of 1.5 μM. Thus, when used under physiological conditions, duplex or triplex formation will be substantially favored over the state in which the antigen and its target are dissociated. It is understood that a stable duplex or triplex may in some embodiments include mismatches between base pairs and/or among base triplets in the case of triplexes. Preferably, modified oligonucleotides, e.g. comprising LNA units, of the invention form perfectly matched duplexes and/or triplexes with their target nucleic acids.

As used herein, the term “downstream” when used in reference to a direction along a nucleotide sequence means in the direction from the 5′ to the 3′ end. Similarly, the term “upstream” means in the direction from the 3′ to the 5′ end.

As used herein, the term “gene” means the gene and all currently known variants thereof and any further variants which may be elucidated.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic”, “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

As used herein, the term “mRNA” means the presently known mRNA transcript(s) of a targeted gene, and any further transcripts which may be elucidated.

By “desired RNA” molecule is meant any foreign RNA molecule which is useful from a therapeutic, diagnostic, or other viewpoint. Such molecules include antisense RNA molecules, decoy RNA molecules, enzymatic RNA, therapeutic editing RNA (Woolf and Stinchcomb, “Oligomer directed In situ reversion (ISR) of genetic mutations”, filed Jul. 6, 1994, U.S. Ser. No. 08/271,280, hereby incorporated by reference) and agonist and antagonist RNA.

By “antisense RNA” is meant a non-enzymatic RNA molecule that binds to another RNA (target RNA) by means of RNA-RNA interactions and alters the activity of the target RNA (Eguchi et al., 1991 Annu. Rev. Biochem. 60, 631-652). By “enzymatic RNA” is meant an RNA molecule with enzymatic activity (Cech, 1988 J. American. Med. Assoc. 260, 3030-3035). Enzymatic nucleic acids (ribozymes) act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through base-pairing, and once bound to the correct site, acts enzymatically to cut the target RNA.

By “decoy RNA” is meant an RNA molecule that mimics the natural binding domain for a ligand. The decoy RNA therefore competes with natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is meant to be a specific example. Those in the art will recognize that this is but one example, and other embodiments can be readily generated using techniques generally known in the art.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 80% or 90%, preferably 95%, most preferably 100%, complementarity to a defined sequence. Preferably, alleles or variants thereof can be identified. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head & neck, kidney, lung, non-small cell lung, melanoma, mesothelioma, ovary, sarcoma, stomach, uterus and Medulloblastoma. The terms “tumor” and “tumor cell” as used herein refer to a cell or aggregation of cells characterized by or resulting from uncontrolled, progressive growth and division of cells. Such cells generally have a deleterious effect on a host organism. A tumor cell may be located in vivo, particularly in a human but also including other animals. A tumor cell may also be located in vitro, and may be treated according to inventive methods and using inventive compositions, for instance for research and/or drug discovery. Inhibition of a tumor cell includes inhibition of growth of such a cell, inhibition of physiological processes, inhibition of metastasis, and preferably includes killing such a cell.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic). Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Examples of sarcomas which can be treated with siRNA's and optionally a potentiator and/or chemotherapeutic agent include, but not limited to a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with siRNA's and optionally a potentiator and/or another chemotherapeutic agent include but not limited to, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas which can be treated with siRNA's and optionally a potentiator and/or a chemotherapeutic agent include but not limited to, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Additional cancers which can be treated with siRNA's according to the invention include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

A “heterologous” component refers to a component that is introduced into or produced within a different entity from that in which it is naturally located. For example, a polynucleotide derived from one organism and introduced by genetic engineering techniques into a different organism is a heterologous polynucleotide which, if expressed, can encode a heterologous polypeptide. Similarly, a promoter or enhancer that is removed from its native coding sequence and operably linked to a different coding sequence is a heterologous promoter or enhancer.

A “promoter,” as used herein, refers to a polynucleotide sequence that controls transcription of a gene or coding sequence to which it is operably linked. A large number of promoters, including constitutive, inducible and repressible promoters, from a variety of different sources, are well known in the art and are available as or within cloned polynucleotide sequences (from, e.g., depositories such as the ATCC as well as other commercial or individual sources).

An “enhancer,” as used herein, refers to a polynucleotide sequence that enhances transcription of a gene or coding sequence to which it is operably linked. A large number of enhancers, from a variety of different sources are well known in the art and available as or within cloned polynucleotide sequences (from, e.g., depositories such as the ATCC as well as other commercial or individual sources). A number of polynucleotides comprising promoter sequences (such as the commonly-used CMV promoter) also comprise enhancer sequences.

“Operably linked” refers to a juxtaposition, wherein the components so described are in a relationship permitting them to function in their intended manner. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence.

A “replicon” refers to a polynucleotide comprising an origin of replication which allows for replication of the polynucleotide in an appropriate host cell. Examples include replicons of a target cell into which a heterologous nucleic acid might be integrated (e.g., nuclear and mitochondrial chromosomes), as well as extrachromosomal replicons (such as replicating plasmids and episomes).

“Gene delivery,” “gene transfer,” and the like as used herein, are terms referring to the introduction of an exogenous polynucleotide (sometimes referred to as a “transgene products”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of gene products to mammalian cells, as is known in the art and described herein.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

A cell is “transduced” by a nucleic acid when the nucleic acid is translocated into the cell from the extracellular environment. Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably replicated. A vector includes a nucleic acid (ordinarily RNA or DNA) to be expressed by the cell. A vector optionally includes materials to aid in achieving entry of the nucleic acid into the cell, such as a viral particle, liposome, protein coating or the like. A “cell transduction vector” is a vector which encodes a nucleic acid capable of stable replication and expression in a cell once the nucleic acid is transduced into the cell.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell.

A “vector” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy. Vectors include, for example, viral vectors (such as adenoviruses (“Ad”), adeno-associated viruses (AAV), and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Viral “packaging” as used herein refers to a series of intracellular events that results in the synthesis and assembly of a viral vector. Packaging typically involves the replication of the “pro-viral genome”, or a recombinant pro-vector typically referred to as a “vector plasmid” (which is a recombinant polynucleotide than can be packaged in an manner analogous to a viral genome, typically as a result of being flanked by appropriate viral “packaging sequences”), followed by encapsidation or other coating of the nucleic acid. Thus, when a suitable vector plasmid is introduced into a packaging cell line under appropriate conditions, it can be replicated and assembled into a viral particle. Viral “rep” and “cap” gene products, found in many viral genomes, are gene products encoding replication and encapsidation proteins, respectively. A “replication-defective” or “replication-incompetent” viral vector refers to a viral vector in which one or more functions necessary for replication and/or packaging are missing or altered, rendering the viral vector incapable of initiating viral replication following uptake by a host cell. To produce stocks of such replication-defective viral vectors, the virus or pro-viral nucleic acid can be introduced into a “packaging cell line” that has been modified to contain gene products encoding the missing functions which can be supplied in trans). For example, such packaging gene products can be stably integrated into a replicon of the packaging cell line or they can be introduced by transfection with a “packaging plasmid” or helper virus carrying gene products encoding the missing functions.

A “detectable marker gene” is a gene that allows cells carrying the gene to be specifically detected (e.g., distinguished from cells which do not carry the marker gene). A large variety of such marker gene products are known in the art. Preferred examples thereof include detectable marker gene products which encode proteins appearing on cellular surfaces, thereby facilitating simplified and rapid detection and/or cellular sorting. By way of illustration, the lacZ gene encoding beta-galactosidase can be used as a detectable marker, allowing cells transduced with a vector carrying the lacZ gene to be detected by staining.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. Selectable markers can be positive, negative or bifunctional. Positive selectable markers allow selection for cells carrying the marker, whereas negative selectable markers allow cells carrying the marker to be selectively eliminated. A variety of such marker gene products have been described, including bifunctional (i.e. positive/negative) markers (see, e.g., WO 92/08796, published May 29, 1992, and WO 94/28143, published Dec. 8, 1994). Such marker gene products can provide an added measure of control that can be advantageous in gene therapy contexts.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The treatment of neoplastic disease or neoplastic cells, refers to an amount of the vectors and/or peptides, described throughout the specification and in the Examples which follow, capable of invoking one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention of tumor cell infiltration into peripheral organs; (6) inhibition, including (i) reduction, (ii) slowing down or (iii) complete prevention of metastasis; (7) enhancement of anti-tumor immune response, which may result in (i) maintaining tumor size, (ii) reducing tumor size, (iii) slowing the growth of a tumor, (iv) reducing, slowing or preventing invasion or (v) reducing, slowing or preventing metastasis; and/or (8) relief, to some extent, of one or more symptoms associated with the disorder.

Treatment of an individual suffering from an infectious disease organism refers to a decrease and elimination of the disease organism from an individual. For example, a decrease of viral particles as measured by plaque forming units or other automated diagnostic methods such as ELISA etc.

As used herein, a “pharmaceutically acceptable” component is one that is suitable for use with humans and/or animals without undue adverse side effects (such as toxicity, irritation, and allergic response) commensurate with a reasonable benefit/risk ratio.

### Compositions

Provided are compositions for inhibiting a tumor cell and for treating an individual having a tumor. The compositions are used in treating an individual having cancer or a tumor and inhibiting a tumor cell are provided according to the present invention.

Nuclear ferritin is present in tumor cells and nuclear localization of the H subunit of ferritin in tumor cells is associated with faster growth and increased survival times in such cells. The presence of ferritin in a nucleus is further associated with a change in gene and protein expression that reflects increased growth that is elevated expression of transcription factors in cell cycle genes. We have shown that the localization of ferritin to the nucleus is dependent on O-glycosylation. Inhibitors of O-glycosylation such as alloxan inhibit translocation of ferritin to the nucleus and thus render cells vulnerable to stressors such as high levels of iron as well as other environmental stressors. Furthermore, we have shown that H ferritin binds DNA with no sequence specific manner. Our results show that H Ferritin protects DNA ftom iron-induced oxidative damage.

Based on our studies, we have found that nuclear ferritin is O-linked glycosylated and non-specific blocking of 0-linked glycosylation decreases the presence of ferritin in the nucleus. The results indicate that ferritin translocation between cytoplasm and nucleus is post-translationally regulated and responds to environmental and nutritional cues. Nuclear ferritin induces changes in gene and protein expression that will increase cellular viability and decrease vulnerability.

Ferritin protects cells from stressors and increases the survival time of astrocytes in culture. The presence of ferritin in the nucleus is associated with a change in the gene and protein expression that reflects increased growth (e.g. elevated expression of transcription factors and cell cycle genes) and decreases the vulnerability of the astrocytes.

A composition is provided according to the present invention which includes an inhibitor of ferritin. In a preferred option, a composition according to the present invention includes an inhibitor of H ferritin. An inhibitor of H ferritin is active to reduce the level of H ferritin protein in a cell and/or to reduce the activity of H ferritin in a cell.

An inhibitor of H ferritin active to reduce the level of H ferritin protein in the cell may be an inhibitor of transcription and/or translation of H ferritin. In addition, an inhibitor of H ferritin active to reduce the level of H ferritin protein in the cell may stimulate degradation of the H ferritin protein and/or H ferritin encoding RNA.

An inhibitor of ferritin transcription and/or translation may be a nucleic acid-based inhibitor such as an antisense oligonucleotides complementary to a target H ferritin mRNA, as well as ribozymes and DNA enzyme which are catalytically active to cleave the target mRNA.

In one embodiment, an inhibitor of H ferritin is small interfering RNA against H ferritin. Particular examples of siRNA directed against H ferritin that may be used include any one of SEQ ID NO's: 1-8.

In another preferred embodiment, siRNA inhibitors of H-ferritin include a combination of SEQ ID NO's: 1 and 2; SEQ ID NO's: 3 and 4; SEQ ID NO's: 5 and 6; SEQ ID NO's: 7 and 8.

In another preferred embodiments, siRNA inhibitors of H-ferritin include combinations of one or more of SEQ ID NO's: 1-8. For example, SEQ ID NO's: 1, 3, 5, 8. Combinations of these inhibitors of H-Ferritin can be used in any fashion needed to inhibit H-ferritins.

In addition to reducing the level of ferritin present in a tumor cell, ferritin's functional characteristics may be interfered with in order to treat an individual having a tumor and inhibit a tumor cell. For instance, the native H ferritin protein may be denatured or bound to another molecule such that H ferritin is less capable of binding iron. Antibodies or other ferritin and/or ferritin subunit binding proteins may be used to bind ferritin, especially H ferritin, inhibiting its functional effects. Further, ferritin may be inhibited by decreasing transport of the protein to a particular subcellular location. In particular, translocation of H ferritin from the cytoplasm to the nucleus of a tumor cell may be decreased in order to inhibit functioning of H ferritin in the nucleus. It is found that nuclear localization of the H subunit of ferritin in tumor cells is associated with faster growth and increased survival times in such cells. The presence of ferritin in a nucleus is further associated with a change in gene and protein expression that reflects increased growth that is elevated expression of transcription factors in cell cycle genes.

Thus, in one embodiment, a method of treating an individual having a tumor and a method of inhibiting a tumor cell are provided which include administering a composition effective to decrease the levels and/or functioning of ferritin, particularly H ferritin, in the nucleus of a tumor cell.

Thus, in one embodiment, a composition according to the present invention includes an inhibitor of translocation of ferritin from the cytoplasm to the nucleus of a tumor cell. In a preferred embodiment, a composition according to the present invention includes an inhibitor of translocation of H ferritin from the cytoplasm to the nucleus of a tumor cell. Such inhibitors include inhibitors of O-glycosylation, such as alloxan.

Further, ferritin may be inhibited by decreasing transport of the protein to a particular subcellular location. In particular, translocation of H ferritin from the cytoplasm to the nucleus of a tumor cell may be decreased in order to inhibit functioning of H ferritin in the nucleus. It is found that nuclear localization of the H subunit of ferritin in tumor cells is associated with faster growth and increased survival times in such cells. The presence of ferritin in a nucleus is further associated with a change in gene and protein expression that reflects increased growth that is elevated expression of transcription factors in cell cycle genes.

Thus, in one embodiment, a method of treating an individual having a tumor and a method of inhibiting a tumor cell are provided which include administering a composition effective to decrease the levels and/or functioning of ferritin, particularly H ferritin, in the nucleus of a tumor cell.

It is shown that the localization of ferritin, particularly H ferritin, to the nucleus is dependent on O-glycosylation. Inhibitors of O-glycosylation such as alloxan inhibit translocation of ferritin to the nucleus and thus render cells vulnerable to stressors such as high levels of iron as well as other environmental stressors.

Thus, in one embodiment, a composition according to the present invention includes an inhibitor of translocation of ferritin from the cytoplasm to the nucleus of a tumor cell. In a preferred embodiment, a composition according to the present invention includes an inhibitor of translocation of H ferritin from the cytoplasm to the nucleus of a tumor cell. Such inhibitors include inhibitors of O-glycosylation, such as alloxan.

In another preferred embodiment, a composition comprises at least on of H-ferritin inhibitors, SEQ ID NO's: 1-8 and an inhibitor of O-glycosylation

Decreasing the level and/or activity of ferritin, especially H ferritin, in a tumor cell, increases the vulnerability of the tumor cell to oxidative stressors. In addition, such treatment renders a tumor cell more vulnerable to the effects of an anti-tumor agent and/or an anti-tumor treatment.

A composition according to the present invention may further include an anti-tumor agent. Exemplary anti-tumor agents include chemotherapeutic compounds such as an antineoplastic, an antimitotic, an antimetabolite, and combinations thereof.

A composition including an agent effective to decrease levels, activity and/or nuclear localization of ferritin, especially H ferritin, along with an anti-tumor agent is particularly advantageous over administration of an anti-tumor agent alone since a synergistic effect of the combined agents may be seen. Thus, the dose of an administered anti-tumor agent in such a composition is lower than would otherwise be required for an anti-tumor effect.

A pharmaceutical delivery system including a particulate delivery vehicle may be used to aid in delivery of a composition according to the present invention to a target cell. For example, such a particulate delivery vehicle may be a liposome. In one example, a particulate delivery vehicle is capable of mediating intracellular delivery of the inhibitor. Further, a particulate delivery vehicle may include a tumor cell targeting moiety such as an antibody, nucleic acid, and/or receptor ligand.

In another preferred embodiment, a pharmaceutical composition is provided which includes an inhibitor of an H ferritin protein and preferably further includes a pharmaceutically acceptable carrier. A pharmaceutically acceptable carrier is generally non-toxic to an individual to be treated in amounts used and does not have deleterious effects on the inhibitor. Such carriers include solvents, buffering agents, preservatives, for example.

A method of treating cancer in an individual having a tumor is provided which includes administration of a composition according to the present invention

Provided methods of treatment of an individual having a tumor optionally further include administration of an anti-tumor treatment. Exemplary anti-tumor treatments include radiation administration including external radiation therapy and/or internal administration of radiation such as by implant radiation. Administration of a composition according to the invention along with an anti-tumor treatment is advantageous over administration of an anti-tumor treatment alone since a synergistic effect of the combined treatments may be seen. Thus, the dose of an administered anti-tumor treatment is lower than would otherwise be required for an anti-tumor effect.

The compositions of the invention may be administered to animals including humans in any suitable formulation. For example, the compositions may be formulated in pharmaceutically acceptable carriers or diluents such as physiological saline or a buffered salt solution. Suitable carriers and diluents can be selected on the basis of mode and route of administration and standard pharmaceutical practice. A description of other exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

In another preferred embodiment, a composition comprising an inhibitor of ferritin is used to treat a patient suffering from iron-related diseases. These disease are characterized by an iron-imbalance, i.e. excess iron or iron-deficiency. Examples of iron-related diseases caused by excess iron include, cancer, neurological disorders, thalassemia, sickle cell anemia and the like. Non-limiting examples are shown below. Inhibitors of ferritin are discussed, infra and include for example, siRNAs, O-glycosylation inhibitors and the like.

| Organ | Disease Or Illness Caused By Excess Iron |
|---|---|
| Liver | Cirrhosis, Liver Cancer |
| Joints/Bones | Osteoarthritis, Osteopenia, Osteomalacia |
| Pancreas | Diabetes |
| Gallbladder | Gallstones |
| Heart | Irregular Heart Beat, Heart Attack |
| Anterior | Hypothyroidism, Infertility, Impotence, |
| Pituitary | Depression, Hypogonadism |
| Skin | Bronze or Ashen Gray Green Color |

In another preferred embodiment, a patient suffering from iron-deficiency related disorders is treated with a composition comprising H-ferritin and/or inducers of H-ferritin. H-ferritin is shown to have protective effects in a cell. See, for example, Surguladze, N. et al (2004) J. Biol. Chem. 279(15):14694-14702, incorporated herein by reference in its entirety. Many of these patients have serious health problems that require multiple treatments such as repeated blood transfusion, iron infusions, iron injections and then iron-chelation therapy to remove the extra iron. Examples of iron-deficiency related diseases include, but not limited to: H. pylori infection, acquired sideroblastic anemia, enzyme disorders such as G6PD deficiency (Glucose-6-phosphate dehydrogenase) and PKD (pyruvate-kinase deficiency). Other diseases can be chronic (ongoing), for example: kidney disease, cancer, thalassemia, sickle cell disease, CDA II (HEMPAS), inherited sideroblastic anemia, MDS (myelodysplasia), porphyria cutanea tarda (PCT), hereditary hemorrhagic telangiectasia (HHT), AIDS, Crohn's, celiac disease, and autoimmune hemolytic anemia's. Treatment, using the compositions of the invention include administration of H-ferritin, e.g. SEQ ID NO: 9 and/or NLS-ferritin, in a pharmaceutical composition and/or delivery vehicle such as a liposome which comprises a targeting moiety such as antibody, receptor, ligand etc. Also within the scope of the invention are use of vectors expressing H-ferritin, e.g. SEQ ID NO: 9 and/or NLS-ferritin under the control of a tissue specific promoter or inducible promoter. The administration of H-ferritin can be combined with one or more other treatments such as EPO (erythropoietin) to stimulate bone marrow.

In addition to reducing the level of ferritin present in a cell such as a tumor cell or cell in which iron fluctuates or is at increased, or decreased levels as compared to a normal cell, ferritin's functional characteristics may be interfered with in order to treat an individual having a tumor and inhibit a tumor cell. For instance, the native H ferritin protein may be denatured or bound to another molecule such that H ferritin is less capable of binding iron. Antibodies or other ferritin and/or ferritin subunit binding proteins may be used to bind ferritin, especially H ferritin, inhibiting its functional effects.

### Inhibition of Gene Expression

Enzymatic nucleic acid molecules (e.g., ribozymes) are nucleic acid molecules capable of catalyzing one or more of a variety of reactions, including the ability to repeatedly cleave other separate nucleic acid molecules in a nucleotide base sequence-specific manner. Such enzymatic nucleic acid molecules can be used, for example, to target virtually any RNA transcript (Zaug et al., 324, Nature 429 1986; Cech, 260 JAMA 3030, 1988; and Jefferies et al., 17 Nucleic Acids Research 1371, 1989).

Because of their sequence-specificity, trans-cleaving enzymatic nucleic acid molecules show promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the mRNA non-functional and abrogates protein expression from that RNA. In this manner, synthesis of a protein associated with a disease state can be selectively inhibited.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J, 9, 1183; Breaker, 1996, Curr. Op. Biotech., 7, 442).

The development of ribozymes that are optimal for catalytic activity would contribute significantly to any strategy that employs RNA-cleaving ribozymes for the purpose of regulating gene expression. The hammerhead ribozyme, for example, functions with a catalytic rate (k_{cat}) of about 1 min⁻¹ in the presence of saturating (10 mM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹. Finally, replacement of a specific residue within the catalytic core of the hammerhead with certain nucleotide analogues gives modified ribozymes that show as much as a 10-fold improvement in catalytic rate. These findings demonstrate that ribozymes can promote chemical transformations with catalytic rates that are significantly greater than those displayed in vitro by most natural self-cleaving ribozymes. It is then possible that the structures of certain self-cleaving ribozymes may be optimized to give maximal catalytic activity, or that entirely new RNA motifs can be made that display significantly faster rates for RNA phosphodiester cleavage.

Intermolecular cleavage of an RNA substrate by an RNA catalyst that fits the “hammerhead” model was first shown in 1987 (Uhlenbeck, O. C. (1987) Nature, 328: 596-600). The RNA catalyst was recovered and reacted with multiple RNA molecules, demonstrating that it was truly catalytic.

Catalytic RNAs designed based on the “hammerhead” motif have been used to cleave specific target sequences by making appropriate base changes in the catalytic RNA to maintain necessary base pairing with the target sequences (Haseloff and Gerlach, Nature, 334, 585 (1988); Walbot and Bruening, Nature, 334, 196 (1988); Uhlenbeck, O. C. (1987) Nature, 328: 596-600; Koizumi, M., Iwai, S. and Ohtsuka, E. (1988) FEBS Lett., 228: 228-230). This has allowed use of the catalytic RNA to cleave specific target sequences and indicates that catalytic RNAs designed according to the “hammerhead” model may possibly cleave specific substrate RNAs in vivo. (see Haseloff and Gerlach, Nature, 334, 585 (1988); Walbot and Bruening, Nature, 334, 196 (1988); Uhlenbeck, O. C. (1987) Nature, 328: 596-600).

RNA interference (RNAi) has become a powerful tool for blocking gene expression in mammals and mammalian cells. This approach requires the delivery of small interfering RNA (siRNA) either as RNA itself or as DNA, using an expression plasmid or virus and the coding sequence for small hairpin RNAs that are processed to siRNAs.

In a preferred embodiment, the invention provides methods for treating tumor cells comprising inhibitors of H-Ferritin. For example, siRNAs comprising any one or more or combinations thereof of SEQ ID NO's: 1-8. Such treatment methods comprise administering a ribozyme-siRNA oligonucleotide and/or siRNAs to tumor cells, including those that comprise an infectious agent, such as Human Papilloma virus (HPV). A variety of cells may be treated in accordance with the compositions and methods of the invention, and typically mammalian cells are treated, especially primate cells such as human cells.

Inhibition of gene expression may be quantified by measuring either the endogenous target RNA or the protein produced by translation of the target RNA. Techniques for quantifying RNA and proteins are well known to one of ordinary skill in the art. In certain preferred embodiments, gene expression is inhibited by at least 10%, preferably by at least 33%, more preferably by at least 50%, and yet more preferably by at least 80%. In particularly preferred embodiments, of the invention gene expression is inhibited by at least 90%, more preferably by at least 95%, or by at least 99% up to 100% within cells in the organism. In preferred embodiments of the invention inhibition occurs rapidly after administration of the compositions of the invention. In preferred embodiments significant inhibition of H-ferritin gene expression occurs within 24 hours after the siRNAs or ribozyme-siRNA comprising any one of, or combinations of SEQ ID NO's: 1-8 is administered to a patient. In more preferred embodiments significant inhibition occurs within 12 hours after administration of the compositions. In yet more preferred embodiments significant inhibition occurs between about 6 to 12 hours after the siRNAs or ribozyme-siRNA comprising any one of, or combinations of SEQ ID NO's: 1-8 is administered to a patient. In yet more preferred embodiments significant inhibition occurs within less than about 6 hours after the siRNAs or ribozyme-siRNA comprising any one of, or combinations of SEQ ID NO's: 1-8 is administered to a patient. By significant inhibition is meant sufficient inhibition to result in a detectable phenotype (e.g., inhibition of viral replication etc.) or a detectable decrease in RNA and/or protein corresponding to the gene being inhibited.

In order to achieve inhibition of a target gene selectively within a given subject which it is desired to control, an RNAi preferably exhibits a high degree of sequence identity with corresponding segments in the subject. Preferably the degree of identity is more than about 80%. Untranslated regions (UTRs), i.e., 5′ and 3′ UTRs, frequently display a low degree of conservation across species since they are not constrained by the necessity of coding for a functional protein. Thus, in certain preferred embodiments the gene portion comprises or includes a UTR. If it is desired to inhibit a target gene within a number of different species which it is desired to control, the RNAi preferably exhibits a high degree of identity with the corresponding segments in these species and a low degree of identity with corresponding nucleic acid sequences in other species, particularly in mammals.

Selection of appropriate RNAi is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of RNAi that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

In a preferred embodiment, small interfering RNA (siRNA) either as RNA itself or as DNA, is delivered to a cell using an expression plasmid or virus and the coding sequence for small hairpin RNAs that are processed to siRNAs. In a preferred embodiment, the siRNAs comprise SEQ ID NO's: 1-8.

In another preferred embodiment, a cloning site for insertion of the hairpin RNA into an expression vector can be in any nucleotide location, as internal base-pairing is preserved in the flanking hammerhead ribozyme (Rz) and hairpin ribozyme.

In accordance with the invention target cells are selectively targeted by an siRNA of SEQ ID NO's: 1-8.

Preferred siRNA's of the invention hybridize (bind) to a target H-Ferritin sequences, under stringency conditions as may be assessed in vitro. Such conditions are disclosed and defined below.

The invention may be used against protein coding gene products as well as non-protein coding gene products. Examples of non-protein coding gene products include gene products that encode ribosomal RNAs, transfer RNAs, small nuclear RNAs, small cytoplasmic RNAs, telomerase RNA, RNA molecules involved in DNA replication, chromosomal rearrangement and the like. In another use for the invention, the siRNA delivery system can be used to target wild-type genes to provide tools for functional genetics or to create cell-based and animal models of genetic disease, such as for example, target validation. Animals of any species, including, but not limited to, mice, rats, rabbits, guinea pigs, pigs, micro-pigs, goats, and non-human primates, e.g., baboons, monkeys, and chimpanzees may be used to generate disease animal models. In addition, cells from humans may be used. These systems may be used in a variety of applications. Such assays may be utilized as part of screening strategies designed to identify agents, such as compounds that are capable of ameliorating disease symptoms. Thus, the animal- and cell-based models may be used to identify drugs, pharmaceuticals, therapies and interventions that may be effective in treating disease and also to understand the mechanics behind diseases.

Cell-based systems may be used to identify compounds that may act to ameliorate disease symptoms. For example, such cell systems may be exposed to a compound suspected of exhibiting an ability to ameliorate disease symptoms, at a sufficient concentration and for a time sufficient to elicit such an amelioration of disease symptoms in the exposed cells. After exposure, the cells are examined to determine whether one or more of the disease cellular phenotypes has been altered to resemble a more normal or more wild type, non-disease phenotype.

In addition, animal-based disease systems, may be used to identify compounds capable of ameliorating disease symptoms. Such animal models may be used as test substrates for the identification of drugs, pharmaceuticals, therapies, and interventions that may be effective in treating a disease or other phenotypic characteristic of the animal. For example, animal models may be exposed to a compound or agent suspected of exhibiting an ability to ameliorate disease symptoms, at a sufficient concentration and for a time sufficient to elicit such an amelioration of disease symptoms in the exposed animals. The response of the animals to the exposure may be monitored by assessing the reversal of disorders associated with the disease. Exposure may involve treating mother animals during gestation of the model animals described herein, thereby exposing embryos or fetuses to the compound or agent that may prevent or ameliorate the disease or phenotype. Neonatal, juvenile, and adult animals can also be exposed.

In another preferred embodiment, abnormal or cancer cells are targeted by the siRNAs. For example, many malignancies are associated with the presence of foreign DNA, e.g. Bcr-Abl, Bcl-2, HPV, and these provide unique molecular targets to permit selective malignant cell targeting.

According to the present invention, an siRNA oligonucleotide is designed to be specific for a molecule, which either causes, participates in, or aggravates a disease state, in a patient.

In accordance with the invention, siRNA oligonucleotide therapies comprise administered siRNA oligonucleotide which contacts (interacts with) the targeted mRNA from the gene, whereby expression of the gene is modulated, and expression is inhibited. Preferably, the siRNAs comprise any one of, or combinations of SEQ ID NO's: 1-8. Such modulation of expression suitably can be a difference of at least about 10% or 20% relative to a control, more preferably at least about 30%, 40%, 50%, 60%, 70%, 80%, or 90% difference in expression relative to a control. It will be particularly preferred where interaction or contact with an siRNA oligonucleotide results in complete or essentially complete modulation of expression relative to a control, e.g., at least about a 95%, 97%, 98%, 99% or 100% inhibition of or increase in expression relative to control. A control sample for determination of such modulation can be comparable cells (in vitro or in vivo) that have not been contacted with the siRNA oligonucleotide.

According to one preferred embodiment of the invention, the nucleobases in the siRNA may be modified to provided higher specificity and affinity for a target mRNA. For example nucleobases may be substituted with LNA monomers, which can be in contiguous stretches or in different positions. The modified siRNA, preferably has a higher association constant (Kₐ) for the target sequences than the complementary sequence. Binding of the modified or non-modified siRNA's to target sequences can be determined in vitro under a variety of stringency conditions using hybridization assays and as described in the examples which follow.

A fundamental property of oligonucleotides that underlies many of their potential therapeutic applications is their ability to recognize and hybridize specifically to complementary single stranded nucleic acids employing either Watson-Crick hydrogen bonding (A-T and G-C) or other hydrogen bonding schemes such as the Hoögsteen/reverse Hoögsteen mode. Affinity and specificity are properties commonly employed to characterize hybridization characteristics of a particular oligonucleotide. Affinity is a measure of the binding strength of the oligonucleotide to its complementary target (expressed as the thermostability (Tₘ) of the duplex). Each nucleobase pair in the duplex adds to the thermostability and thus affinity increases with increasing size (No. of nucleobases) of the oligonucleotide. Specificity is a measure of the ability of the oligonucleotide to discriminate between a fully complementary and a mismatched target sequence. In other words, specificity is a measure of the loss of affinity associated with mismatched nucleobase pairs in the target.

The utility of an siRNA oligonucleotide for modulation (including inhibition) of an mRNA, e.g. H-ferritin can be readily determined by simple testing. Thus, an in vitro or in vivo expression system comprising the targeted mRNA, mutations or fragments thereof, can be contacted with a particular siRNA oligonucleotide (modified or un modified) and levels of expression are compared to a control, that is, using the identical expression system which was not contacted with the siRNA oligonucleotide. This is described in detail in the examples which follow.

siRNA oligonucleotides may be used in combinations. For instance, a cocktail of several different siRNA modified and/or unmodified oligonucleotides, directed against different regions of the same gene, may be administered simultaneously or separately. For example, the vector expressing the ribozyme siRNA cassette may comprise one or a plurality of such cassettes in tandem, for example, 2, 3, 4 etc.

Chimeric/Modified siRNA Cassettes

In accordance with this invention, persons of ordinary skill in the art will understand that mRNA includes not only the coding region which carries the information to encode a protein using the three letter genetic code, including the translation start and stop codons, but also associated ribonucleotides which form a region known to such persons as the 5′-untranslated region, the 3′-untranslated region, the 5′ cap region, intron regions and intron/exon or splice junction ribonucleotides. Thus, oligonucleotides may be formulated in accordance with this invention which are targeted wholly or in part to these associated ribonucleotides as well as to the coding ribonucleotides. In preferred embodiments, the oligonucleotide is targeted to a translation initiation site (AUG codon) or sequences in the coding region, 5′ untranslated region or 3′-untranslated region of an mRNA.

In a preferred embodiment, the siRNAs comprise any one or more of SEQ ID NO's: 1-8 and/or combinations thereof. The functions of messenger RNA to be interfered include all vital functions such as translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with protein expression.

Certain preferred oligonucleotides of this invention are chimeric oligonucleotides. “Chimeric oligonucleotides” or “chimeras”, in the context of this invention, are oligonucleotides which contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the RNA target) and a region that is a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art. In one preferred embodiment, a chimeric oligonucleotide comprises at least one region of SEQ ID NO's: 1-8, modified to increase target binding affinity, and, usually, a region that acts as a substrate for RNAse H. Affinity of an oligonucleotide for its target (in this case, a nucleic acid encoding ras) is routinely determined by measuring the Tₘ of an oligonucleotide/target pair, which is the temperature at which the oligonucleotide and target dissociate; dissociation is detected spectrophotometrically. The higher the Tₘ, the greater the affinity of the oligonucleotide for the target. In a more preferred embodiment, the region of the oligonucleotide which is modified comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrymidines, abasic residues or an inverted base at the 3′ end of the RNA. Such modifications are routinely incorporated into oligonucleotides and these oligonucleotides have been shown to have a higher Tₘ (i.e., higher target binding affinity) than; 2′-deoxyoligonucleotides against a given target. The effect of such increased affinity is to greatly enhance RNAi oligonucleotide inhibition of gene expression. RNAse H is a cellular endonuclease that cleaves the RNA strand of RNA:DNA duplexes; activation of this enzyme therefore results in cleavage of the RNA target, and thus can greatly enhance the efficiency of RNAi inhibition. Cleavage of the RNA target can be routinely demonstrated by gel electrophoresis. In another preferred embodiment, the chimeric oligonucleotide is also modified to enhance nuclease resistance. Cells contain a variety of exo- and endo-nucleases which can degrade nucleic acids. A number of nucleotide and nucleoside modifications have been shown to make the oligonucleotide into which they are incorporated more resistant to nuclease digestion than the native oligodeoxynucleotide. Nuclease resistance is routinely measured by incubating oligonucleotides with cellular extracts or isolated nuclease solutions and measuring the extent of intact oligonucleotide remaining over time, usually by gel electrophoresis. Oligonucleotides which have been modified to enhance their nuclease resistance survive intact for a longer time than unmodified oligonucleotides. A variety of oligonucleotide modifications have been demonstrated to enhance or confer nuclease resistance. Oligonucleotides which contain at least one phosphorothioate modification are presently more preferred. In some cases, oligonucleotide modifications which enhance target binding affinity are also, independently, able to enhance nuclease resistance. Some desirable modifications can be found in De Mesmaeker et al. Acc. Chem. Res. 1995, 28:366-374.

Specific examples of some preferred oligonucleotides envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, —N(CH₃)—O—CH₂ [known as a methylene(methylimino) or MMI backbone], CH₂—O—N (CH₃)—CH₂, CH₂—N(CH₃)—N(CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also preferred. Also preferred are oligonucleotides having morpholino backbone structures (Summerton and Weller, U.S. Pat. No. 5,034,506). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). Oligonucleotides may also comprise one or more substituted sugar moieties. Preferred oligonucleotides comprise one of the following at the 2′ position: OH, SH, SCH₃, F, OCN, OCH₃ OCH₃, OCH₃ O(CH₂)ₙCH₃, O(CH₂)ₙNH₂ or O(CH₂)ₙCH₃ where n is from 1 to about 10; C₁ to C₁₀ lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂ CH₂ OCH₃, also known as 2′-O-(2-methoxyethyl)] (Martin et al., Helv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W.H. Freeman & Co., San Francisco, 1980, pp 75-77; Gebeyehu, G., et al. Nucl. Acids Res. 1987, 15:4513). A “universal” base known in the art, e.g., inosine, may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). Oligonucleotides comprising lipophilic moieties, and methods for preparing such oligonucleotides are known in the art, for example, U.S. Pat. Nos. 5,138,045, 5,218,105 and 5,459,255.

It is not necessary for all positions in a given oligonucleotide to be uniformly modified, and in fact more than one of the aforementioned modifications may be incorporated in a single oligonucleotide or even at within a single nucleoside within an oligonucleotide. The present invention also includes oligonucleotides which are chimeric oligonucleotides as hereinbefore defined.

In another embodiment, the nucleic acid molecule of the present invention is conjugated with another moiety including but not limited to abasic nucleotides, polyether, polyamine, polyamides, peptides, carbohydrates, lipid, or polyhydrocarbon compounds. Those skilled in the art will recognize that these molecules can be linked to one or more of any nucleotides comprising the nucleic acid molecule at several positions on the sugar, base or phosphate group.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of one of ordinary skill in the art. It is also well known to use similar techniques to prepare other oligonucleotides such as the phosphorothioates and alkylated derivatives. It is also well known to use similar techniques and commercially available modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other modified oligonucleotides such as cholesterol-modified oligonucleotides.

In accordance with the invention, use of modifications such as the use of LNA monomers to enhance the potency, specificity and duration of action and broaden the routes of administration of oligonucleotides comprised of current chemistries such as MOE, ANA, FANA, PS etc (ref: Recent advances in the medical chemistry of antisense oligonucleotide by Uhlman, Current Opinions in Drug Discovery & Development 2000 Vol 3 No 2). This can be achieved by substituting some of the monomers in the current oligonucleotides by LNA monomers. The LNA modified oligonucleotide may have a size similar to the parent compound or may be larger or preferably smaller. It is preferred that such LNA-modified oligonucleotides contain less than about 70%, more preferably less than about 60%, most preferably less than about 50% LNA monomers and that their sizes are between about 10 and 25 nucleotides, more preferably between about 12 and 20 nucleotides.

In a preferred embodiment, siRNAs comprising at least one or more of SEQ ID NO's: 1-8 or combinations thereof are administered with one or more inhibitors of H-Ferritin as described herein and/or combined with other therapies for treatment of tumors such as chemotherapy.

Delivery of siRNA

Preferred invention practice involves administering at least one of the foregoing siRNA polynucleotides with a suitable particulate or nucleic acid delivery system. In one embodiment, that system includes a non-viral vector operably linked to the polynucleotide. Examples of such non-viral vectors include the polynucleoside alone or in combination with a suitable protein, polysacchalide or lipid formulation.

Additionally suitable nucleic acid delivery systems include viral vector, typically sequence from at least one of an adenovirus, adenovirus-associated virus (AAV), helper-dependent adenovirus, retrovirus, or hemagglutinating virus of Japan-liposome (HVJ) complex. Preferably, the viral vector comprises a strong eukaryotic promoter operably linked to the polynucleotide e.g., a cytomegalovirus (CMV) promoter.

Additionally preferred vectors include viral vectors, fusion proteins and chemical conjugates. Retroviral vectors include moloney murine leukemia viruses and HIV-based viruses. One preferred HIV-based viral vector comprises at least two vectors wherein the gag and poT genes are from an HIV genome and the env gene is from another virus. DNA viral vectors are preferred. These vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum Gene Ther 4:151 (1993)) and MMT promoters may also be used. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely effect the metabolism of the organism being treated. The cassette comprising any one or more of SEQ ID NO's: 1-8 can also be bound to a nucleic acid binding moiety in a synthetic delivery system, such as the system disclosed in WO 95/22618.

If desired, the polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989). Liposomes can comprise a targeting moiety such as for example an antibody, receptor and/or ligand and the like.

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another preferred siRNA delivery method is to use single stranded DNA producing vectors which can produce the siRNA's intracellularly. See for example, Chen et al, BioTechniques, 34: 167-171 (2003), which is incorporated herein, by reference, in its entirety.

The effective dose of the nucleic acid will be a function of the particular expressed protein, the particular cardiac arrhythmia to be targeted, the patient and his or her clinical condition, weight, age, sex, etc.

One preferred delivery system is a recombinant viral vector that incorporates one or more of the polynucleotides therein, preferably about one polynucleotide. Preferably, the viral vector used in the invention methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful e.g., about 1 nanogram to about 100 micrograms.

### Promoters

In a preferred embodiment, the expression cassette comprising one or more or combinations thereof, of SEQ ID NO's: 1-8 is expressed and processed by RNA polymerases. Throughout this application, the term “expression construct” is meant to include any type of genetic construct containing a nucleic acid coding for gene products in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding genes of interest.

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase. Much of the thinking about how promoters are organized derives from analyses of several viral promoters, including those for the HSV thymidine kinase (tk) and SV40 early transcription units. These studies, augmented by more recent work, have shown that promoters arc composed of discrete functional modules, each consisting of approximately 7-20 b.p. of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 b.p. upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 b.p. apart before activity begins to decline. Depending on the promoter, it appears that individual elements can function either co-operatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system would be preferable so that the producer cells could be grown in the presence of tetracycline or doxycycline and prevent expression of a potentially toxic transgene, but when the vector is introduced to the patient, the gene expression would be constitutively on.

In some circumstances, it may be desirable to regulate expression of a transgene in a gene therapy vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic Virus, HSV-TK, and avian sarcoma virus.

In a preferred embodiment, tissue specific promoters are used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, the following promoters may be used to target gene expression in other tissues (Table 1).

TABLE 1
Tissue specific promoters

| Tissue | Promoter |
|---|---|
| Pancreas | insulin |
| | elastin |
| | amylase |
| | pdr-1 pdx-1 |
| | glucokinase |
| Liver | albumin PEPCK |
| | HBV enhancer |
| | alpha fetoprotein |
| | apolipoprotein C |
| | alpha-1 antitrypsin |
| | vitellogenin, NF-AB |
| | Transthyretin |
| Skeletal muscle | myosin H chain |
| | muscle creatine kinase |
| | dystrophin |
| | calpain p94 |
| | skeletal alpha-actin |
| | fast troponin 1 |
| Skin | keratin K6 |
| | keratin K1 |
| Lung | CFTR |
| | human cytokeratin 18 (K18) |
| | pulmonary surfactant proteins A, B and C |
| | CC-10 |
| | P1 |
| Smooth muscle | sm22 alpha |
| | SM-alpha-actin |
| Endothelium | endothelin-1 |
| | E-selectin |
| | von Willebrand factor |
| | TIE |
| | KDR/flk-1 |
| Melanocytes | tyrosinase |
| Adipose tissue | lipoprotein lipase |
| | adipsin |
| | acetyl-CoA carboxylase |
| | glycerophosphate dehydrogenase |
| | adipocyte P2 |
| Blood | β-globin |

In certain indications, it may be desirable to activate transcription at specific times after administration of the gene therapy vector. This may be done with such promoters as those that are hormone or cytokine regulatable. For example in gene therapy applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful in the present invention. Cytokine and inflammatory protein responsive promoters that could be used include K and T Kininogen, c-fos, TNF-alpha, C-reactive protein, haptoglobin, serum amyloid A2, C/EB.P. alpha, IL-1, IL-6, Complement C3, IL-8, alpha-1 acid glycoprotein, alpha-1 antitypsin, lipoprotein lipase, angiotensinogen, fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

It is envisioned that cell cycle regulatable promoters may be useful in the present invention. For example, in a bi-cistronic gene therapy vector, use of a strong CMV promoter to drive expression of a first gene such as p16 that arrests cells in the G1 phase could be followed by expression of a second gene such as p53 under the control of a promoter that is active in the G1 phase of the cell cycle, thus providing a “second hit” that would push the cell into apoptosis. Other promoters such as those of various cyclins, PCNA, galectin-3, E2F1, p53 and BRCA1 could be used.

Tumor specific promoters such as osteocalcin, hypoxia-responsive element (HRE), MAGE-4, CEA, alpha-fetoprotein, GRP78/BiP and tyrosinase may also be used to regulate gene expression in tumor cells. Other promoters that could be used according to the present invention include Lac-regulatable, chemotherapy inducible (e.g. MDR), and heat (hyperthermia) inducible promoters, radiation-inducible (e.g., EGR), Alpha-inhibin, RNA pol III tRNA met and other amino acid promoters, U1 snRNA, MC-1, PGK, β-actin and α-globin. Many other promoters that may be useful are known in the art.

It is envisioned that any of the above promoters alone or in combination with another may be useful according to the present invention depending on the action desired. In addition, this list of promoters is should not be construed to be exhaustive or limiting, those of skill in the art will know of other promoters that may be used in conjunction with the promoters and methods disclosed herein.

### Assessing Gene Silencing

Transfer of an exogenous nucleic acid into a host cell or organism by a vector can be assessed by directly detecting the presence of the nucleic acid in the cell or organism. Such detection can be achieved by several methods well known in the art. For example, the presence of the exogenous nucleic acid can be detected by Southern blot or by a polymerase chain reaction (PCR) technique using primers that specifically amplify nucleotide sequences associated with the nucleic acid. Expression of the exogenous nucleic acids can also be measured using conventional methods. For instance, mRNA produced from an exogenous nucleic acid can be detected and quantified using a Northern blot and reverse transcription PCR (RT-PCR).

Expression of an RNA from the exogenous nucleic acid can also be detected by measuring an enzymatic activity or a reporter protein activity. For example, siRNA activity can be measured indirectly as a decrease in target nucleic acid expression as an indication that the exogenous nucleic acid is producing the effector RNA.

### Target Validation

The nucleic acid molecules of the present invention can inhibit gene expression in a highly specific manner and prevent production of the gene product (Christoffersen, Nature Biotech, 1997, 2, 483-484). Appropriate delivery vehicles can be combined with these nucleic acid molecules (including polymers, cationic lipids, liposomes and the like) and delivered to appropriate cell culture or in vivo animal disease models as described above. By monitoring inhibition of gene expression and correlation with phenotypic results, the relative importance of the particular gene sequence to disease pathology can be established. The process can be both fast and highly selective, and allow for the process to be used at any point in the development of the organism. The novel chemical composition of these nucleic acid molecules can allow for added stability and therefore increased efficacy.

### Candidate Therapeutic Agents

In a preferred embodiment, a method of identifying candidate therapeutic compounds comprises culturing cells comprising an inhibitor of H-ferritin, such as for example, siRNAs comprising one or more or combinations thereof of SEQ ID NO's: 1-8, with a candidate therapeutic agent; identifying candidate therapeutic agents which inhibit proliferation or growth, and/or lyse the cells; wherein, growth and/or metastasis of a tumor is inhibited, and identifying a candidate therapeutic agent. Preferably, a candidate therapeutic agent comprises organic molecules, inorganic molecules, vaccines, antibodies, nucleic acid molecules, proteins, peptides and vectors expressing nucleic acid molecules.

In another preferred embodiment, a method of identifying candidate therapeutic compounds comprises culturing cells, such as for example, tumor cells, with an inhibitor of H-Ferritin O-glycosylation, such as alloxan.

Once a compound that inhibits tumors is identified, the compound can be considered a candidate compound that inhibits tumor formation, such as for example, inhibit cell proliferation, growth, stem cell migration and the like. The ability of such compounds to treat tumors can be evaluated in a population of viable cells or in an animal, e.g., an animal model.

Such compounds are useful, e.g., as candidate therapeutic compounds for the treatment of cancer. Thus, included herein are methods for screening for candidate therapeutic compounds for the treatment of, for example, cancer. The methods include administering the compound to a model of the condition, e.g., contacting a cell (in vitro) model with the compound, or administering the compound to an animal model of the condition, e.g., an animal model of a condition associated with decreased stem cell migration, such as cancer. The model is then evaluated for an effect of the candidate compound on the rate of migration in the model, and a candidate compound that decreases the rate of migration in the model can be considered a candidate therapeutic compound for the treatment of the condition. Such effects can include clinically relevant effects such as decreased tumor size or decreased tumor growth rate; decreased metastatic involvement or decreased rate of metastasis; decreased pain; increased life span; and so on. Such effects can be determined on a macroscopic or microscopic scale. Candidate therapeutic compounds identified by these methods can be further verified, e.g., by administration to human subjects in a clinical trial.

Test Compounds: The test compounds utilized in the assays and methods described herein can be, inter alia, nucleic acids, small molecules, organic or inorganic compounds, antibodies or antigen-binding fragments thereof, polynucleotides, peptides, or polypeptides. For example, polypeptide variants including truncation mutants, deletion mutants, and point mutants; nucleic acids including sense, antisense, aptamers, and small inhibitory RNAs (siRNAs) including short hairpin RNAs (shRNAs) and ribozymes) can be used as test compounds in the methods described herein. Alternatively, compounds or compositions that antagonize H-ferritin mechanisms of action can be used, e.g. inhibit nuclear translocation, glycosylation and the like can be used. A test compound that has been screened by an in vitro method described herein and determined to have a desired activity, or affecting the functions of H-Ferritin cytoprotective effects, for example, affecting growth, proliferation, and the like, can be considered a candidate compound. A candidate compound that has been screened, e.g., in an in vitro or in vivo model, and determined to have a desirable effect on one or more inhibitory activities associated with treatment of cancer, (e.g. inhibition of proliferation, anti-angiogenic, apoptosis, decreased cell growth etc) can be considered a candidate therapeutic agent. Candidate therapeutic agents, once screened in a clinical setting, are therapeutic agents, and both types of agents can be optionally optimized (e.g., by derivatization), and formulated with pharmaceutically acceptable excipients or carriers to form pharmaceutical compositions.

Small Molecules: Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

In some embodiments, the compounds are optimized to improve their therapeutic index, i.e., increase therapeutic efficacy and/or decrease unwanted side effects. Thus, in some embodiments, the methods described herein include optimizing the test or candidate compound. In some embodiments, the methods include formulating a therapeutic composition including a test or candidate compound (e.g., an optimized compound) and a pharmaceutically acceptable carrier. In some embodiments, the compounds are optimized by derivatization using methods known in the art.

RNA Interference (RNAi): RNAi is a remarkably efficient process whereby double-stranded RNA (dsRNA, also referred to herein as siRNAs, for small interfering RNAs, or ds siRNAs, for double-stranded small interfering RNAs) induces the sequence-specific degradation of homologous mRNA in animals and plant cells (Hutvagner and Zamore, Curr. Opin. Genet. Dev., 12:225-232 (2002); Sharp, Genes Dev., 15:485-490 (2001)). In mammalian cells, RNAi can be triggered by duplexes of small interfering RNA (siRNA) (Chiu et al., Mol. Cell., 10:549-561 (2002); Elbashir et al., Nature, 411:494-498 (2001)), or by micro-RNAs (miRNA), functional small-hairpin RNA (shRNA), or other dsRNAs which are expressed in vivo using DNA templates with RNA polymerase III promoters (Zeng et al., Mol. Cell, 9:1327-1333 (2002); Paddison et al., Genes Dev., 16:948-958 (2002); Lee et al., Nature Biotechnol., 20:500-505 (2002); Paul et al., Nature Biotechnol., 20:505-508 (2002); Tuschl, T., Nature Biotechnol., 20:440-448 (2002); Yu et al., Proc. Natl. Acad. Sci. USA, 99(9):6047-6052 (2002); McManus et al., RNA, 8:842-850 (2002); Sui et al., Proc. Natl. Acad. Sci. USA, 99(6):5515-5520 (2002)).

The methods described herein can include the use of dsRNA molecules that are targeted to (i.e., bind to) H-ferritin, e.g. any one or more, or combinations thereof of SEQ ID NO: 1-8.

The dsRNA molecules typically comprise 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is identical or substantially identical to the first strand. Each strand can also have one or more overhanging (i.e., non-complementary) nucleotides, e.g., one, two, three, four or more overhanging nucleotides, e.g., dTdTdT.

The dsRNA molecules can be chemically synthesized, or can be transcribed in vitro from a DNA template, or in vivo from, e.g., shRNA. The dsRNA molecules can be designed using any method known in the art; a number of algorithms are known in the art, see, e.g., Tuschl et al., Genes Dev 13(24):3191-7 (1999), and many are available on the internet, e.g., on the websites of Dharmacon (Lafayette, Colo.) or Ambion (Austin, Tex.).

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

micro RNA (miRNAs) of approximately 22 nucleotides can be used to regulate gene expression at the post transcriptional or translational level. miRNAs can be excised in the cell from an approximately 70 nucleotide precursor RNA stem-loop by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng (2002), supra). When expressed by DNA vectors containing polymerase III promoters, micro-RNA designed hairpins can silence gene expression (McManus (2002), supra).

dsRNA can be delivered directly into cells in vivo or in vitro using methods known in the art, e.g., cationic liposome transfection, nanoparticles, and electroporation, or expressed in vivo or in vitro from recombinant DNA constructs that allow longer-term target gene suppression in cells, including mammalian Pol III promoter systems (e.g., H1 or U6/snRNA promoter systems (Tuschl (2002), supra) capable of expressing functional double-stranded siRNAs; (Bagella et al., J. Cell. Physiol. 177:206-213 (1998); Lee et al. (2002), supra; Miyagishi et al. (2002), supra; Paul et al. (2002), supra; Yu et al. (2002), supra; Sui et al. (2002), supra).

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Transcriptional termination by RNA Pol III occurs at runs of four consecutive T residues in the DNA template, providing a mechanism to end the siRNA transcript at a specific sequence. The dsRNA thus produced is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into cells with a vector expression T7 RNA polymerase (Jacque (2002), supra).

In an animal, whole-embryo electroporation can efficiently deliver synthetic siRNA into post-implantation mouse embryos (Calegari et al., Proc. Natl. Acad. Sci. USA, 99(22): 14236-40 (2002)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)). Local delivery can also be used, e.g., with a carrier such as lipiodol (iodine in oil) to facilitate delivery into cells.

Engineered RNA precursors, introduced into cells or whole organisms as described herein, can be used for the production of a desired siRNA molecule. Such an siRNA molecule can then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the protein encoded by that mRNA in the cell or organism. Additional information regarding the use of RNAi can be found in RNA Interference Editing, and Modification: Methods and Protocols (Methods in Molecular Biology), Gott, Ed. (Humana Press, 2004);

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

Embodiments of inventive compositions and methods are illustrated in the following examples. This example is provided for illustrative purposes and is not considered a limitation on the scope of inventive compositions and methods.

### EXAMPLES

### Example 1

### Synergistic Effects of H-Ferritin Inhibitors and Chemotherapeutic Drug

Glioma cells lines SW1088 and U251 are used in vitro to demonstrate a synergistic effect of H ferritin inhibition and a chemotherapeutic drug agent to produce a cytotoxic effect on tumor cells.

The cells are transfected with siRNA against H ferritin or control nucleic acid as described in the appended pages. At 48 hours after transfection, a chemotherapeutic anti-tumor agent is added at varying concentrations to cells. In this example the chemotherapeutic agents Temodar and BCNU are used. Effectiveness of this treatment is measured by determining the number of dead cells present at a time following treatment with the chemotherapeutic agent. An MTS assay is used to assess the number of dead cells.

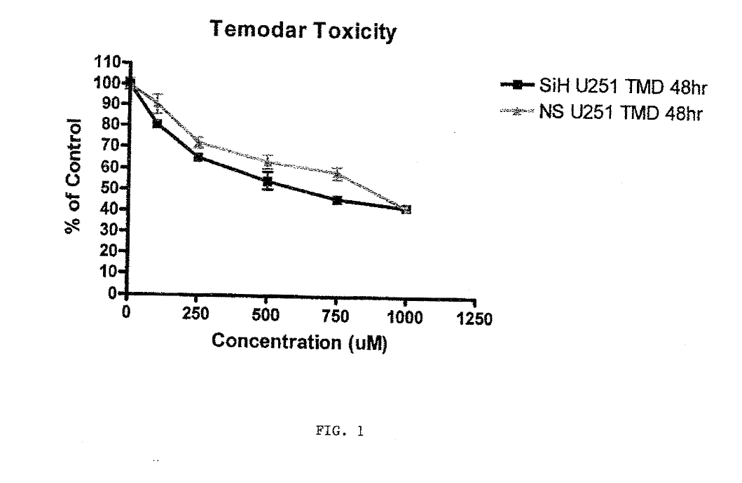

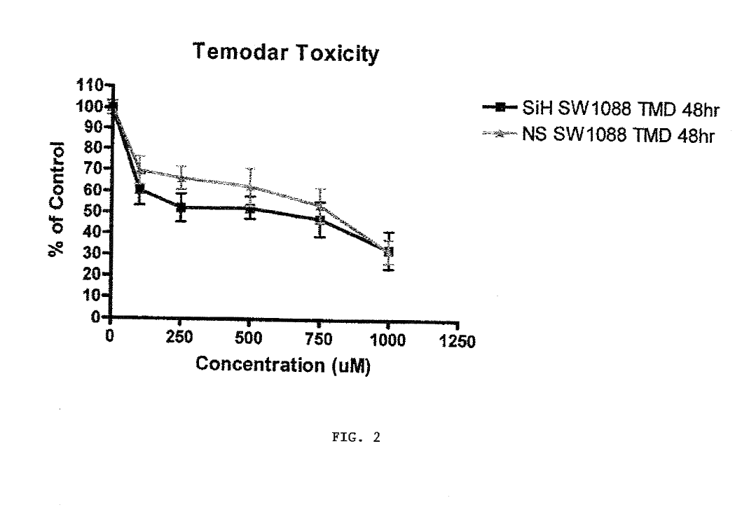

FIGS. 1 and 2 illustrate the effects of siRNA against H ferritin in combination with Temodar (blue triangles) on U251 and SW1088 cells, respectively, compared to control RNA sequences in combination with Temodar (black rectangles) on the same cells.

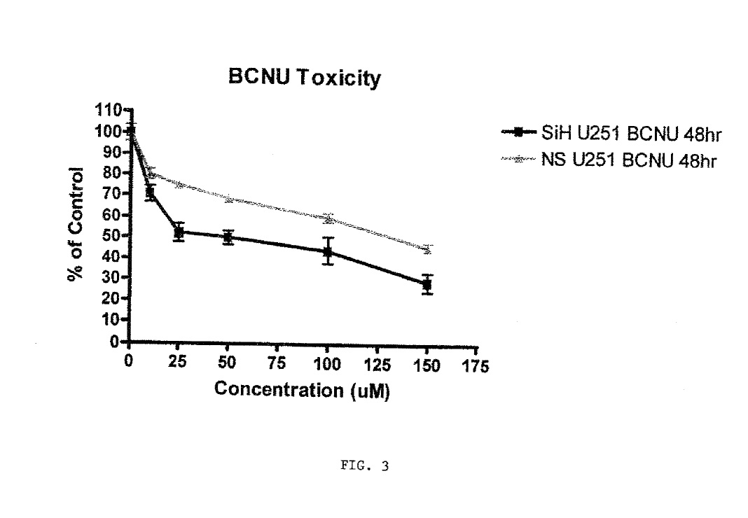

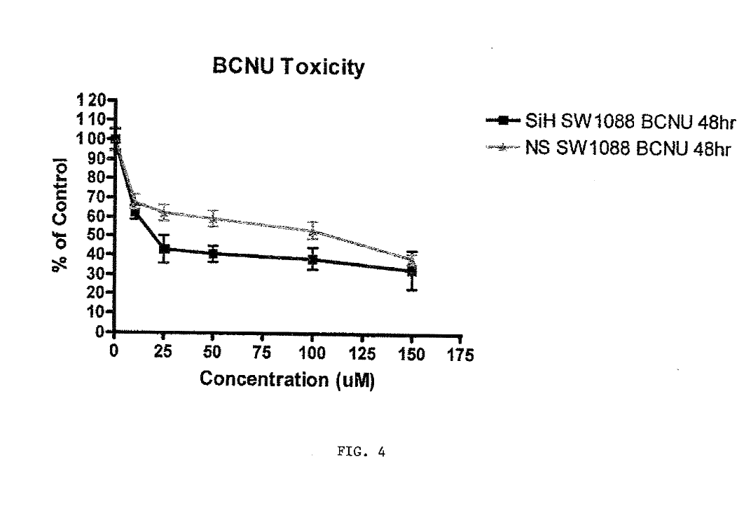

FIGS. 3 and 4 show the effects of siRNA against H ferritin in combination with BCNU (blue triangles) on U251 and SW1088 cells, respectively, compared to control RNA sequences in combination with BCNU (black rectangles) on the same cells.

### Example 2

### Nuclear Ferritin and Mechanism of Translocation

Abbreviations used: AEBSF, 4-(2-aminoethyl)benzenesulphonyl fluoride; DAPI, 4,6-diamidino-2-phenylindole; DFO, desferoxamine; DMEM, Dulbecco's modified Eagle's medium; DTT, dithiothreitol; E-64, trans-epoxysuccinyl-L-leucylamido-(4-guanidino)butane; FAC, ferric ammonium citrate; LDH, lactate dehydrogenase; MTT, 3-(4,5-dimethylthiazol-2-yl)-2,5-diphenyl-2H-tetrazolium bromide; NLS, nuclear localization signal; O-GlcNAc, O-linked N-acetylglucosamine; siRNA, small interfering RNA.

Reagents and antibiotics: DFO (desferoxamine), alloxan, the vital stain azure C, DAPI (4,6-diamidino-2-phenylindole), L-glutamine and the protease inhibitors AEBSF [4-(2-aminoethyl)benzenesulphonyl fluoride], aprotinin, leupeptin, bestatin, pepstatin and E-64 [trans-epoxysuccinyl-L-leucylamido-(4-guanidino)butane] were obtained from Sigma. Penicillin, streptomycin and trypsin were from Gibco BRL (Gaithersburg, Md., U.S.A.). All other biochemicals were of the reagent grade.

Cell culture: Human astrocytoma SW1088 cells (A.T.C.C., Manassas, Va., U.S.A.) were cultured in supplemented DMEM [Dulbecco's modified Eagle's medium supplemented with 10% (v/v) fetal calf serum (Biocell, Cardiff, U.K.), 4 mM L-glutamine, 100 units/ml penicillin and 1 ng/ml streptomycin]. Cultures were maintained in 150 mm culture flasks and passaged every 7 days.

Cell viability assay: Cell viability measurement was performed using the reagent 3-(4,5-dimethylthiazol-2-yl)-2,5-diphenyl-2H-tetrazolium bromide (MTT), obtained from Roche Applied Science (Indianapolis, Ind., U.S.A.). The assay was performed according to the manufacturer's instructions.

DFO and alloxan treatments: The iron chelator DFO was used to examine the effect of iron chelation on ferritin localization in the nuclei of astrocytoma cells. Cells were cultured in the presence of 100 μM DFO for 72 h. Alloxan, an inhibitor of O-glycosylation, was used in astrocytoma cell cultures at final concentrations of 0.1 and 1.0 mM. Six cultures were established in parallel and allowed to reach 80% confluence. The first three were treated with 100 μM DFO for 72 h, then rinsed in Hanks balanced salt solution to remove the free DFO and returned to standard medium alone or to a medium containing 0.1 mM FAC (ferric ammonium citrate) or 0.1 mM DFO. After these treatments, culturing was continued for 24 h. Another set of three cultures were treated with 100 μM DFO+1 mM alloxan for 72 h. The cells were rinsed in Hanks balanced salt solution to remove free reagents and returned to standard medium containing 1 mM alloxan or a medium containing 0.1 mM FAC+0.1 mM alloxan or a medium containing 100 μM DFO+1 mM alloxan. After these treatments, the culturing was continued for 24 h. At the end of the culture period, cells were harvested and nuclear and cytosolic fractions were prepared as described below.

Nuclear and cytosolic fractions: Plated astrocytoma cells were rinsed in Hanks balanced salts, trypsinized and the cells were collected by centrifugation (800 g for 10 min). The pelleted cells were rinsed twice in ice-cold PBS buffer [0.01 M sodium phosphate (pH 7.4 at 25° C.), 138 mM NaCl and 2.7 mM KCl] and collected by centrifugation. The cell pellets were resuspended in 20 vol. of 10 mM Hepes (pH 7.9 at 25° C.), 1.5 mM MgCl₂, 10 mM NaCl and 0.5 mM DTT (dithiothreitol) containing the following protease inhibitors: AEBSF (1 mM), aprotinin (0.8 μM), leupeptin (20 μM), bestatin (40 μM), pepstatin (15 μM) and E-64 (14 μM). Cell suspensions were incubated in ice for 30 min and collected by centrifugation. The pelleted cells were resuspended in 10 vol. of 10 mM Hepes (pH 7.9 at 25° C.), 1.5 mM MgCl₂, 10 mM NaCl and 0.5 mM DTT containing 0.5% Nonidet P40 and homogenized gently using a Dounce homogenizer. Cell lysis was verified by light microscopy. Nuclei were collected by centrifugation at 1000 g for 10 min. The supernatant was collected for cytosolic analysis. Crude nuclei were resuspended by gentle homogenization in 0.88 M sucrose and 3 mM MgCl₂ and centrifuged at 2500 g for 20 min to remove cell debris. The pellet was collected, resuspended in the buffer appropriate for nucleoli, nuclear matrix or soluble chromatin preparation (see below) and stored at −80° C. until use. Assay for the cytoplasmic marker enzyme LDH (lactate dehydrogenase) was performed on all nuclear fractions to determine cytoplasmic contamination (Graham, J. M. (1993) Methods Mol. Biol. 19, 1-18).

Soluble nuclear fraction: Isolated nuclei were washed once with 0.1 mM PBS containing 0.1% Triton X-100 and resuspended in hypo-osmotic 10 mM Hepes (pH 7.0), 150 mM sucrose and 10 mM NaCl. The resulting suspension was centrifuged for 10 min at 3000 g. The supernatant was retained as a soluble nuclear fraction.

Digestion with nuclease: Isolated nuclei were washed once with 0.1 mM PBS containing 0.1% Triton X-100 and resuspended in 10 mM Hepes (pH 7.0), 150 mM sucrose, 10 mM NaCl and 1.5 mM MgCl₂. Digestion with DNase I (100 units/μg of nucleic acid) or DNase I+RNase A (1 unit/μg of nucleic acid) was performed at 37° C. for 10 min. The resulting suspension was centrifuged for 10 min at 3000 g. The supernatant was retained as a nuclease-digested soluble nuclear fraction.

Nuclear matrix: Isolated nuclei were washed once with 0.1 mM PBS containing 0.1% Triton X-100 and resuspended in 10 mM Hepes (pH 7.0), 150 mM sucrose, 50 mM NaCl and 3 mM MgCl₂ for digestion with DNase I (100 units/μg of DNA). Digestion was performed at 37° C. for 10 min. Nuclei were collected by centrifugation at 800 g for 10 min at 4° C. and suspended in high-salt-containing buffer at 4° C. in 10 mM Hepes (pH 7.4 at 4° C.), 2 M NaCl, 1 mM EGTA and 300 mM sucrose. The nuclease-resistant matrix fraction was collected by centrifugation at 10000 g for 15 min.

Nucleoli: Isolated nuclei were suspended by gentle homogenization in 0.34 M sucrose and 0.5 mM MgCl₂, transferred to an ice-cold sonicator rosette and sonicated (10×10 s bursts followed by 20 s cooling periods). The release of nucleoli was monitored by microscopic examination and staining with Azure C (Bush, E. (1967) Nucleic acids. In Methods in Enzymology, vol. 12 (Grossman, L. and Moldave, K., eds.), pp. 448-464, Academic Press, New York). Sonicated fractions were underlayered with 5 vol. of 0.88 M sucrose and centrifuged at 3000 g for 20 min. The pellet contained isolated nucleoli.

Production of siRNAs (small interfering RNAs): Generation and purification of the double-stranded siRNAs has been described previously (Cozzi, A. et al. (2004) Blood 103, 2377-2283). The sequences used in the present study are shown in Table 2. Anti-sense siRNAs were obtained from Qiagen (Chatsworth, Calif., U.S.A.) for use as experimental controls.

TABLE 2

Sequences of double-stranded siRNAs
used to suppress H-ferritin translation

| | |
|---|---|
| siRNA | Sequence |

| | | |
|---|---|---|
| H-siRNA1 (beginning 29 nt | 5′-GCCAGAACUACCACCAGGAC-3′ | (SEQ ID NO: 1) |
| downstream of start codon) | 3′-CGGUCUUGAUGGUGGUCCUG-5′ | (SEQ ID NO: 2) |
| H-siRNA2 (beginning 129 nt | 5′-GUGGCUUUGAAGAACUUUGC-3′ | (SEQ ID NO: 3) |
| downstream of start codon) | 3′-CACCGAAACUUCUUGAAACG-5′ | (SEQ ID NO: 4) |
| H-siRNA3 (beginning 333 nt | 5′-GAAUCAGUCACUACUGGAAC 3′ | (SEQ ID NO: 5) |
| downstream of start codon) | 3′-CUUAGUCUGUGAUGACCUUG-5′ | (SEQ ID NO: 6) |
| H-siRNA4 (beginning 501 nt | 5′-GGAAUAUCUCUUUGACAAGC-3′ | (SEQ ID NO: 7) |
| downstream of start codon) | 3′-CCUUAUAGAGAAACUGUUCG-5′ | (SEQ ID NO: 8) |

Proteins and antibodies: The H-ferritin-specific antibodies (rHO2 and HS-59) and recombinant H-ferritin (rH-ferritin) used in the present study have been described previously (Cavanna, F. et al. (1983) Clin. Chim. Acta 134, 347-356; Cavanna, F. et al. (1984) Ric. Clin. Lab. 14, 337-340; Luzzago, A. et al. (1986) Biochim. Biophys. Acta 872, 61-71; Cazzola, M. et al. (1985) Br. J. Haematol. 61, 445-453). Polyclonal anti-human ferritin antibody (special order, Quality Controlled Biochemicals, Hopkinton, Mass., U.S.A.) and human liver ferritin (lot no. 23) were purchased from ICN (Aurora, Ohio, U.S.A.). A monoclonal antibody raised against O-GlcNAc (O-linked N-acetylglucosamine) was purchased from Covance and Alexa 488-conjugated goat anti-mouse IgG was from Molecular Probes.

Cell transfection, immunofluorescence and confocal microscopy. Double-stranded siRNAs (1.4 mg) were transfected into 2×10⁶ SW1088 cells using the Amaxa Rat Astrocyte Nucleofector™ kit (Amaxa, Gaithersburg, Md., U.S.A.) according to the manufacturer's instructions. The resulting cells were grown in DMEM (Gibco BRL), 10% (v/v) fetal bovine serum (ClonTech Laboratories, Palo Alto, Calif., U.S.A.), 100 units/ml penicillin, 100 μg/ml streptomycin and 1 mM L-glutamine. Cells were harvested at approx. 80% confluency. Analysis of a parallel control transfection of these cells, with the pc3.1-GFP (where GFP stands for green fluorescent protein) construct (provided by Amaxa), showed that approx. 75% of the cells were transfected. As a negative control, SW1088 cells were mock-transfected under the same conditions using water in place of the siRNA solution.

As a further test of transfection efficiency and as a control for non-specific effects of the transfection procedure, SW1088 cells were transfected with a rhodamine-conjugated non-specific siRNA (obtained from Qiagen). After 24 h in culture, cells were harvested and suspended in an equal volume of lysis buffer [20 mM Hepes (pH 7.4 at 21° C.), 4% (w/v) SDS, 20 mM DTT, 1 mM AEB SF, 0.8 μM aprotinin, 20 μM leupeptin, 40 μM bestatin, 15 μM pepstatin and 14 μM E-64]. Cells were sonicated for 10 s on ice, protein concentrations were measured and ferritin contents were detected by Western blotting using an anti-human H-ferritin monoclonal antibody (HS-59).

After transfection, approx. 5×10⁴ cells were plated on untreated glass slips. Cells were allowed to attach for 1 h at 37° C. under a 5% humid CO₂ atmosphere; subsequently, the slips were submerged in supplemented DMEM and cultured at 37° C. under a 5% humid CO₂ atmosphere. At 24 h intervals, slips were removed, washed three times with PBS and fixed in ice-cold acetone for 3 min. The slips were incubated for 2 h at room temperature (21° C.) with 10% (v/v) normal goat serum (Vector Laboratories, Burlingame, Calif., U.S.A.) to block non-specific interactions; the slips were then incubated with primary anti-H monoclonal (rH02) or polyclonal antibodies (in 5% normal goat serum) for 90 min at room temperature. Free antibodies were removed by three washes with PBS and the slips were then incubated with Alexa-conjugated goat anti-mouse or, where appropriate, anti-rabbit secondary antibody and DAPI for a further 60 min. After three washes with PBS, slips were mounted and covered with a coverslip. Negative controls for antibody staining were obtained by preparing the slips as described above, but omitting the primary antibody. Confocal microscopy was performed using a Leica TCS equipped with a DMR inverted microscope and a ×63/1.4 NA objective. An argon/krypton laser was used to generate light at 488 nm for Alexa excitation and 360 nm for DAPI excitation. A high-pass filter with a wavelength cutoff at 500 nm was used to recover Alexa fluorescence and a low-pass filter with wavelength cutoff at 460 nm was used to detect DAPI fluorescence. Each fluorophore was excited sequentially to achieve the lowest interference between the two detector channels.

Fluorimetric analysis: Digital pictures were taken at ×10 magnification with a 2-s aperture using a Cooke Sensicam digital camera to obtain a baseline level of fluorescence. For each time period, three different slips were examined and, within each slip, multiple (≧3) microscopic fields were captured for analysis. Nuclear ferritin content was analyzed on the basis of the fluorescence intensities of the entire nuclear regions. Images were analyzed for mean density pixel counts per bright object with ImagePro 4.5 software (Phase Imaging Systems, Glen Mills, Pa., U.S.A.). Background fluorescence was determined and subtracted from each image. The results are presented normalized to a control value obtained with nuclei subjected to mock transfection using a buffer instead of siRNA.

Immunoprecipitation and Western-blot analyses: Cell lysate samples (1 ml, ˜500 μg of protein) were incubated with 20 ml of Protein A/G-agarose beads (Pierce, Rockford, Ill., U.S.A.) for 1 h and then centrifuged at 800 g for 3 min at 4° C. Supernatants were collected and incubated with a polyclonal antibody raised against human ferritin or against the O-glucosyl-moiety for 2 h at 4° C. A further 20 μl of Protein A/G-agarose beads was then added and the mixture was incubated overnight at 4° C. The pellets were collected by centrifugation at 800 g for 5 min at 4° C. and washed by suspension in PBS buffer followed by centrifugation. This procedure was repeated four times. Pellets were then suspended in SDS-gel loading buffer, boiled for 5 min and analyzed by SDS/PAGE. After electrophoresis, proteins were transferred on to 0.2 μm nitrocellulose membranes using a Bio-Rad Trans-Blot apparatus. Membranes were incubated overnight with solutions of HS-59 mouse anti-rH-ferritin antibody or rabbit anti-human ferritin polyclonal antibody at 1:1500 dilution. Immunocomplexes were detected using peroxidase-conjugated goat anti-mouse or anti-rabbit IgGs (Sigma), visualized with the ECL+kit (Amersham Biosciences, Piscataway, N.J., U.S.A.). Images were captured using Blue Bio Film (Denville Scientific, Metuchen, N.J., U.S.A.). Relative amounts of immunoproducts were estimated by film densitometry using ImageQuan™ software (Molecular Dynamics, Sunnyvale, Calif., U.S.A.) as described by Surguladze et al. (Surguladze, N., Thompson, K. M., Beard, J. L., Connor, J. R. and Fried, M. G. (2004) J. Biol. Chem. 279, 14694-14702).

Detection of glycosylated proteins: SW1088 cultures were treated with different concentrations of alloxan for 96 h. At the end of the culture period, cells were harvested, resuspended in an equal volume of lysis in buffer and sonicated for 10 s on ice, as described above. Protein concentrations were measured and aliquots containing 1, 5 and 10 μg of total cell proteins were applied in triplicate to a 0.2 μm nitrocellulose membrane (Amersham Biosciences). Membranes were blocked by incubating for 1 h at 21±1° C. with 5% blotto and then incubated overnight with solutions of monoclonal anti-GlcNAc antibody at 1:1000 dilution. Immunocomplexes were detected using peroxidase-conjugated goat anti-mouse IgG (Sigma) and visualized with the ECL+kit (Amersham Biosciences). Images were captured using Blue Bio Film (Denville Scientific). Relative amounts of the immunoproducts were estimated by film densitometry using ImageQuant™ software (Molecular Dynamics) by the method of Surguladze et al. (2004).

Supercoil relaxation assay: DNA backbone breakage was detected using a superhelical DNA relaxation assay (Tachon, P. (1989) Free Radical Res. Commun. 7, 1-10; Crowe, A. and Morgan, E. H. (1992) Brain Res. 592, 8-16). The reaction mixtures (30 μl) contained covalently closed circular pUC19 DNA (0.5 μg; Sigma) dissolved in 10 mM Hepes (pH 7.5), 50 mM NaCl, 2.5 mM MgCl₂ and 2.5 mM DTT. This particular preparation of DNA has a range of superhelical densities that allow the resolution of topoisomers under our standard electrophoresis conditions, in the absence of added ethidium bromide. With other preparations, only a single band (representing the ensemble of closed-circular topoisomers) and one band representing the relaxed form are resolved. Reactions were initiated by the addition of recombinant human H-ferritin (rH-ferritin). After timed incubations, reactions were terminated by the addition of 0.1 vol. of 50% (v/v) glycerol, 50 mM EDTA and 0.1% Bromophenol Blue. Samples were subjected to electrophoresis on 1.5% (w/v) agarose gels. The mole fractions of superhelical and relaxed forms were measured by densitometry of photographic negatives of the gels after staining with 0.5 μg/ml ethidium bromide.

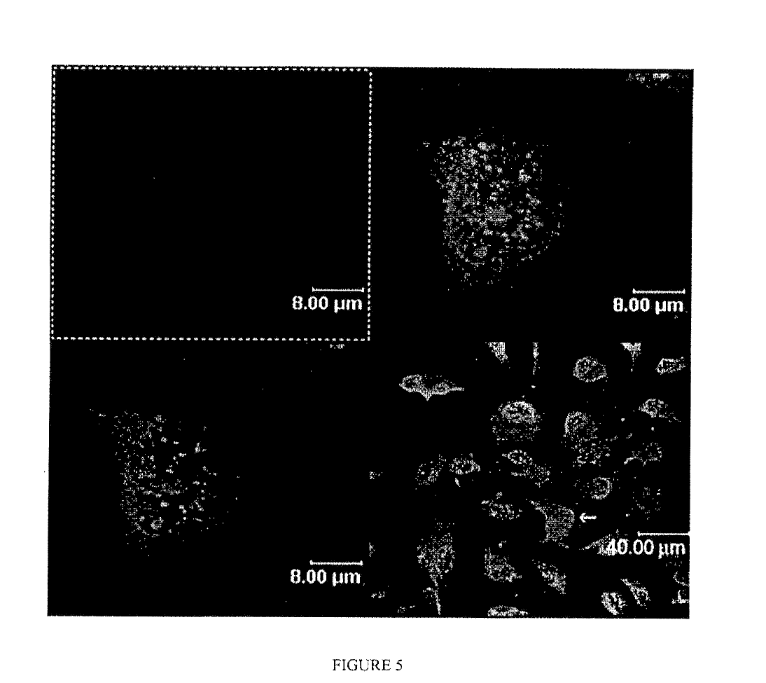

Ferritin is unevenly distributed within the nuclear volume: We have used confocal microscopy to evaluate the intranuclear distribution of ferritin in cultured astrocytoma cells (FIG. 5). Optical thin slices through cells stained with Alexa-labeled antiferritin antibody and counterstained with DAPI reveal that ferritin is present in both nuclear and perinuclear regions. In both compartments, the distribution of ferritin appears to be non-uniform, with discrete regions of intense staining, surrounded by regions with almost no staining. Within the nuclear volume, the size of the intensely stained regions is highly variable, ranging from nearly 1 μm down to the limit of detection. There is no immunocytochemically detectable L-ferritin in the cell nuclei.

The non-uniform distribution of H-ferritin might be due to its association with a particular nuclear component. Fractionation studies were therefore performed to determine the distribution of ferritin between bulk chromatin (solubilized with DNase I), nucleoli and nuclear matrix. Cultured SW1088 cells, which contain nuclear ferritin, were used for these studies. The cytoplasmic enzyme LDH was assayed to detect contamination by cytoplasmic components. The nuclear preparations used in these studies contained 1% of the LDH activity of cytoplasmic fractions.

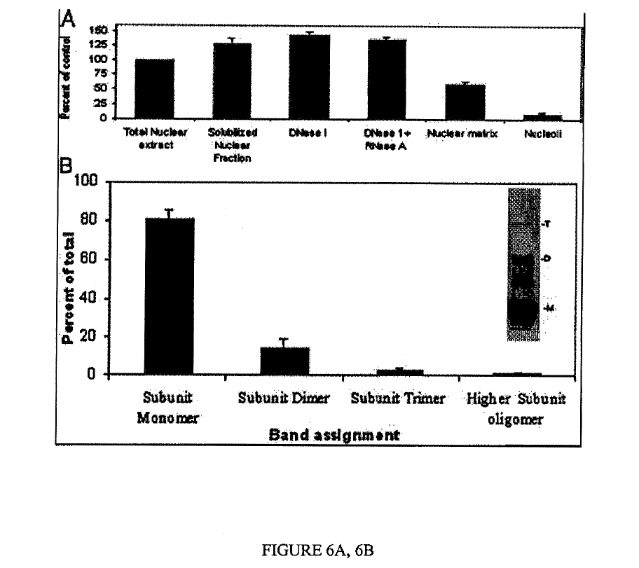

Relative to total protein, the highest amount of ferritin was found in soluble nuclear fractions, an intermediate quantity was found in the nuclear matrix and the smallest amount was found in nucleoli (FIG. 6A). The non-uniform distribution of ferritin in these fractions is consistent with the pattern of nuclear staining described above and suggests that H-ferritin may be preferentially associated with one or more components of heterochromatin.

Ferritin release is not enhanced by DNase action: Several lines of evidence indicate that some forms of H-ferritin but not L-ferritin are capable of binding DNA. We reasoned that if ferritin interacts preferentially with exposed DNA or with extended chromatin regions, it should be preferentially released by the action of DNase I. (FIG. 6A). When nuclei were subjected to protein extraction with and without DNase I, the inclusion of DNase did not increase the concentration of ferritin present in the soluble fraction as detected by immunoblotting. This result suggests that ferritin may not be preferentially associated with DNase I-sensitive structures or that any association is too labile to be detected by these methods. The dynamic nature of ferritin-DNA interactions in vitro is consistent with the latter view.

The ferritin that is present in SW1088 nuclei appears to exist in several covalent states (FIG. 6B). SDS/PAGE analysis with Western-blot detection shows that approx. 85% of the molecules migrated with a relative molecular mass Mᵣ of approx. 22000, consistent with the monomer molecular mass of human H ferritin deduced from the sequence (Mᵣ 21094). Approximately 10% of the protein had an M_{r of} 45000, suggesting the presence of covalent subunit dimers. The remainder (˜5%) migrated more slowly, suggesting the presence of even more subunit multimers. L-ferritin was not detected in the SDS/PAGE analysis consistent with the immunocytochemical results.

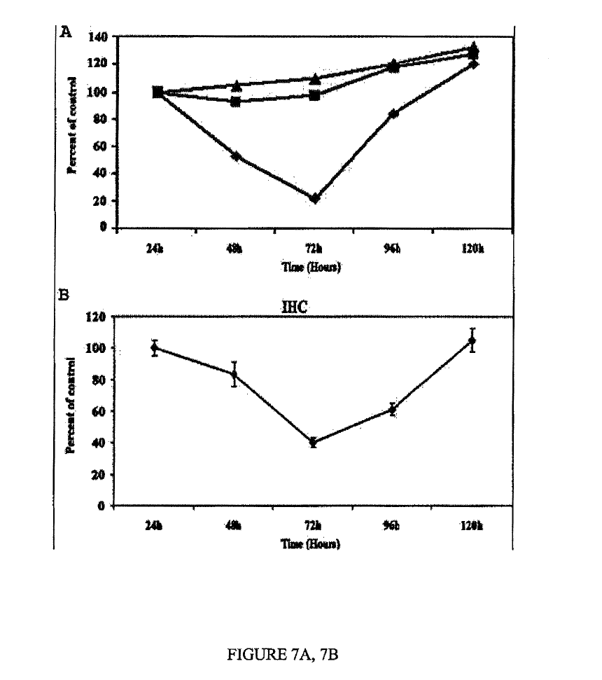

Nuclear and cytoplasmic H-ferritins appear to be products of the same gene: The results presented above demonstrate the presence of a ferritin-like nuclear protein of appropriate molecular mass (Mᵣ˜21000) that reacts efficiently with mouse and rabbit anti-human H-ferritin antibodies. Other experiments indicate that this protein resists thermal denaturation and attack by proteinase K in a manner that is characteristic of cytoplasmic ferritin. However, these results do not establish that the nuclear protein is identical with the cytoplasmic form of ferritin. To determine whether the nuclear and cytoplasmic forms of ferritin are products of a common gene, siRNA inhibition of translation was performed. siRNAs were prepared against DNA sequences encoding cytoplasmic H-ferritin (Table 1). These were transfected into SW1088 cells and the relative ferritin concentrations in nuclear and cytoplasmic compartments were determined as a function of time after transfection. As shown in FIG. 7A, the H-ferritin nuclear signal was significantly decreased within 48 h of transfection with H-ferritin siRNA. The relative amount of nuclear ferritin continued to decrease until 72 h after transfection, after which the signal gradually returned to normal by the 144 h time point. Cells transfected with a non-specific RNA showed no decrease in ferritin (FIG. 7B). Taken together, these results suggest that both nuclear and cytoplasmic H-ferritins are products of the same gene.

The siRNA-dependent changes in cytoplasmic ferritin concentration are mirrored by those in the nucleus, although the changes in nuclear concentration take place with an approx. 16 h delay. This delay suggests that nuclear ferritin concentrations depend on those in the cytoplasm and is consistent with models in which ferritin is synthesized in the cytoplasm and subsequently transferred to the nucleus.

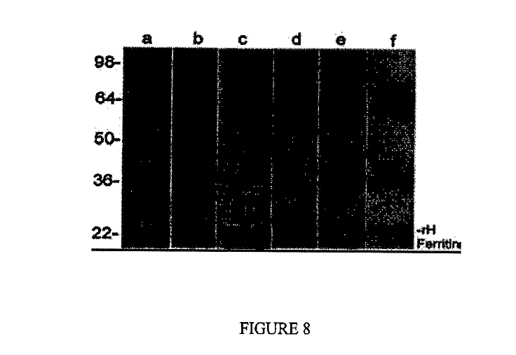

Detection of glycosylated H-ferritin. If the cytoplasmic and nuclear forms of H-ferritin are products of the same gene, what regulates the distribution of the protein between nuclear and cytoplasmic compartments? One possibility is O-glycosylation. Analysis of the H-ferritin sequence using the program YinOYang 1.2 (Center for Biological Sequence Analysis, Technical University of Denmark; available at http://www.cbs.dtu.dk/researchgroups/protfunction.php) predicts the existence of six sites for O-glycosylation, whereas the sequence of L-ferritin (which appears to be restricted to the cytoplasm) has only one site for O-glycosylation (Table 3). To determine whether nuclear and cytoplasmic H-ferritins differ in O-glycosylation, fractions were isolated from SW1088 cells and subjected to immunoprecipitation using antibodies raised against O-GlcNAc. The immunoprecipitated proteins were analyzed by PAGE and Western blotting using a polyclonal H-ferritin antibody as the detection reagent (FIG. 8). Strong signals with electrophoretic mobilities consistent with Mᵣ˜22000 support our conclusion that O-GlcNAc-H-ferritin is present in both nuclear and cytoplasmic fractions.

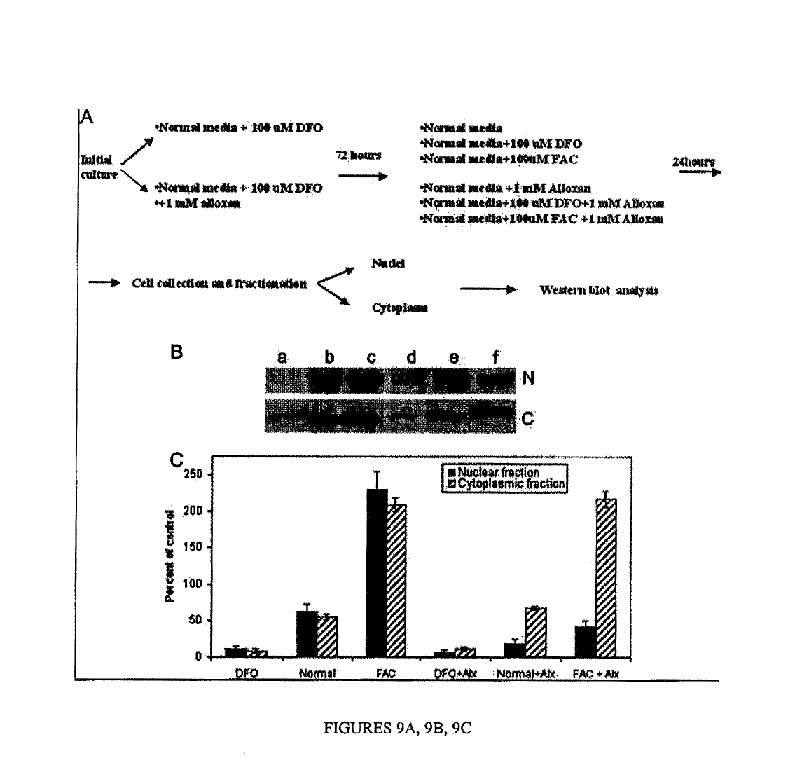

Inhibition of O-glycosylation also inhibits translocation of H-ferritin: To determine whether the O-linked glycosylation was functionally involved in ferritin nuclear translocation, cells were treated with alloxan, a strong inhibitor of O-linked glycosylation. SW1088 cells contain nuclear ferritin under resting conditions. Therefore to study the effect of alloxan on ferritin translocation, we first treated the cells with the iron chelator DFO to decrease the amount of nuclear ferritin. Cells were cultured for 3 days either in normal medium supplemented with 100 μM DFO or in 100 μM DFO+1 mM alloxan. After this initial growth period, the cells cultured with DFO alone were grown for 72 h in either fresh medium, a medium supplemented with 100 μM DFO or a medium supplemented with 100 μM FAC. The cells initially cultured in the presence of DFO and alloxan were grown for 72 h in fresh medium containing 1 mM alloxan, in fresh medium supplemented with 100 μM DFO+1 mM alloxan or in fresh medium supplemented with 100 μM FAC+1 mM alloxan. This experimental procedure is schematically represented in FIG. 9A. In experiments in which cells were treated with DFO in the absence of alloxan, ferritin immunostaining reappeared in nuclei after the cells were returned to standard media (FIG. 9B). If alloxan was present in the initial growth period, the reappearance of nuclear ferritin was inhibited, while cytoplasmic ferritin remained essentially unchanged. Adding iron (FAC) to the standard medium resulted in an approx. 200% increase in nuclear and cytoplasmic ferritin contents relative to levels found in untreated cells. However, the presence of alloxan in the FAC-supplemented medium blocked the increase in nuclear ferritin (FIG. 9B) but not the increase of cytoplasmic ferritin (FIG. 9C).

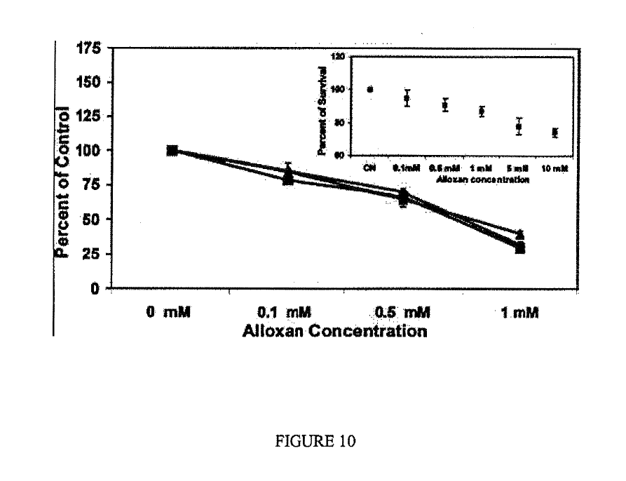

The effect of alloxan on ferritin redistribution is probably due to its influence on protein glycosylation or due to some other currently unknown effect. To demonstrate that alloxan inhibits O-glycosylation in SW1088 cell cultures, cells were treated with 0, 0.1, 0.5 and 1.0 mM alloxan for 96 h. The cells were then harvested and total soluble proteins were analyzed by slot blot for O-glycosylation. The results are shown in FIG. 10 and indicate that alloxan treatment decreases the total amount of O-glycosylated protein in a dose-dependent manner. The concentration of alloxan chosen did not affect cell viability (inset to FIG. 10). Together, these results support the hypothesis that ferritin O-glycosylation and translocation into the nucleus are coupled processes.

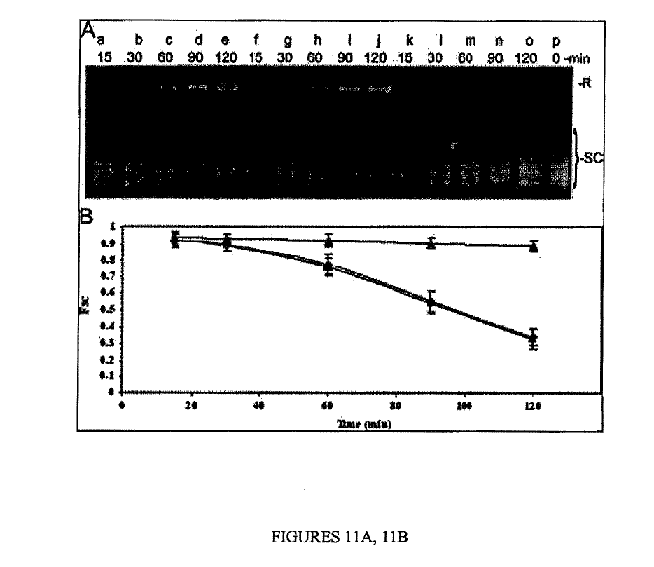

Alloxan treatment does not induce iron release from ferritin. To determine whether alloxan-induced iron release from ferritin is a possible explanation for the differences in ferritin translocation to the nucleus, evidence for alloxan-induced iron release was sought using the in vitro DNA supercoil-relaxation assay. We have shown previously that iron released from ferritin rapidly nicks superhelical DNA and that this provides a sensitive assay for iron release. Supercoil-relaxation assays were performed with the plasmid pUC 19 DNA incubated with ferritin in the presence of different concentrations of alloxan. Comparison of the time course of reactions run with rH-ferritin and rH-ferritin+alloxan shows that all reactions have a characteristic lag at early times, followed by an interval of increasing nicking activity (FIGS. 11A, 11B). The duration of the lag phase and the steady-state nicking rates are similar for all reactions with or without alloxan present, strongly suggesting that, under our assay conditions, alloxan treatment does not effect the rate or amount of iron release from ferritin.

TABLE 3
Putative O-glycosylation sites on H- and L-ferritin subunits
Predicted by the Center for Biological Sequence Analysis
(CBS) online prediction server: YinOYang 1.2.

| | Residue | | | Threshold | |
|---|---|---|---|---|---|
| Sequence | result | O-GlcNAc | Potential threshold | (1) | (2) |
| H-ferritin | 1 T | +++ | 0.5374 | 0.3210 | 0.3831 |
| | 2 T | +++ | 0.7013 | 0.3336 | 0.4000 |
| | 4 S | +++ | 0.7070 | 0.3357 | 0.4029 |
| | 5 T | ++ | 0.5910 | 0.3643 | 0.4414 |
| | 6 S | ++ | 0.4751 | 0.3778 | 0.4597 |
| | 178 S | + | 0.3927 | 0.3202 | 0.3820 |
| | 182 S | + | 0.3243 | 0.2905 | 0.3420 |
| L-ferritin | 1 S | ++ | 0.4819 | 0.3371 | 0.4146 |
| | 2 S | +++ | 0.5466 | 0.3443 | 0.4048 |
| | 9 S | + | 0.4223 | 0.3797 | 0.4622 |
| | 10 T | + | 0.4564 | 0.3837 | 0.4676 |

Discussion: We have shown that ferritin isolated from the nuclei of SW1088 astrocytoma cells shares many properties with cytoplasmic human H-ferritin, including mobility in SDS/PAGE, resistance to proteolysis and heat denaturation and reaction with polyclonal and monoclonal antibodies raised against the cytoplasmic form. Furthermore, siRNA directed against the mRNA-encoding cytoplasmic human H-ferritin decreases the presence of ferritin in SW1088 nuclei in a transient manner. Thus, unlike mitochondrial ferritin, which appears to be transcribed from a different gene, nuclear ferritin is translated from the same mRNA as cytoplasmic ferritin. The L-ferritin subunit was not detected in the nuclear fraction, consistent with previous publications on these cells from our laboratory (Thompson, K. J., Fried, M. G., Ye, Z., Boyer, P. and Connor, J. R. (2002) J. Cell Sci. 115, 2165-2177) and also on other cell types by others. What is the source of the nuclear protein? Initial studies have suggested that cytoplasmic ferritin diffuses into the cell nucleus. More recently, we have shown that ferritin can enter the nuclei through the nuclear pore. Our current hypothesis is that ferritin molecules partition between cytoplasmic and nuclear pools in response to environmental and developmental cues.

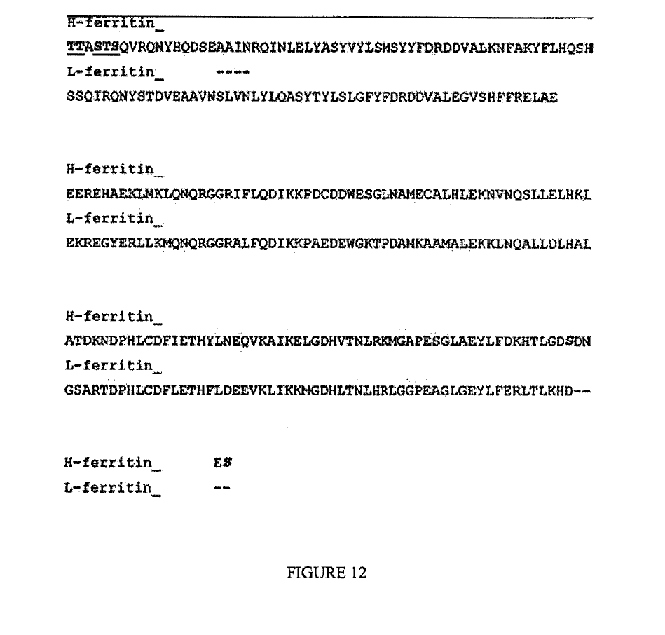

What controls the distribution of H-ferritin between nuclear and cytoplasmic compartments? The lack of any known nuclear localization sequences suggests that a different signal may regulate its translocation. One possibility is O-glycosylation. Monoclonal antibodies or lectins that bind O-GleNAc block the nuclear transport of macromolecules at an energy-dependent step. We have previously shown that movement of ferritin into the nucleus is ATP-dependent and can be blocked by wheatgerm agglutinin. A theoretical search for possible O-glycosylation sites revealed four high-probability sites located in the N-terminal end of the H-ferritin sequence (Table 3) that are not present in the L-ferritin primary sequence (FIG. 12). Only one site was predicted for L-ferritin (Table 3). The prediction that H-ferritin is O-glycosylated is supported experimentally: ferritin was efficiently immunoprecipitated from nuclear and cytoplasmic fractions with antibodies raised against O-GlcNAc (FIG. 8).

The difference in the number and location of potential O-glycosylation sites between H- and L-ferritin suggests a rationale for the preferential nuclear uptake of H-ferritin. Our results indicate that total ferritin is distributed between the cytoplasm and nucleus in a 6:1 molar ratio, but the ratio is decreased more than 3-fold (1.8:1) when the relative amounts of O-glycosylated ferritin are considered (FIG. 8). These results suggest that the molar ratio of ferritin that is O-glycosylated is much higher in the nucleus than in the cytoplasm, consistent with the notion that O-glycosylation is necessary for H-ferritin transfer to the nucleus.

Treatment of SW1088 cells with alloxan decreases the glycosylation of soluble proteins without significant effects on cell viability and blocks the DFO-dependent translocation of ferritin from the cytoplasm to the nucleus. This effect is amplified when cells are grown in the presence of FAC, which significantly increases the nuclear ferritin content (FIGS. 9A-9C and 10). Taken together, these results indicate that glycosylation plays an important role in ferritin translocation. However, at present, we do not know whether alloxan exerts its effect on translocation by blocking ferritin glycosylation or by inhibiting the glycosylation of some other component that is essential for ferritin translocation (e.g. a subunit of the nuclear pore complex). In view of these effects, it is intriguing that alloxan does not prevent the disappearance of ferritin from the nuclear fraction when DFO is added. This result indicates that the effect of alloxan on the concentration of ferritin in the nucleus is asymmetric: it inhibits ferritin uptake but not its removal.

Alloxan had no effect on the reappearance of ferritin in cytoplasmic fractions when DFO-treated cells were returned to normal medium. The presence of FAC in the growth medium did not change this outcome even though cells contained approx. two times more ferritin when cultured with this iron source than when grown in its absence. In contrast with the trend seen with nuclear fractions, the presence of alloxan had no detectable effect on the ferritin content of cytoplasmic fractions. Together, these results indicate that alloxan treatment has little or no effect on the synthesis of detectable, cytoplasmic H-ferritin species or on the depletion of ferritin from the cytoplasm in the presence of strong chelators such as DFO.

Confocal microscopy performed on the immunohistochemically stained cells reveals a heterogeneous distribution of ferritin within the nuclei of SW1088 cells (FIG. 5). A similar ‘speckled’ appearance has been reported for ferritin in the nuclei of K562 cells. Together, these results suggest that ferritin is concentrated in specific nuclear volumes (as distinct from a uniform distribution). Ferritin may be associated with one or more components that form foci or ferritin passively occupies volumes around and between other nuclear structures. Both are consistent with the ‘punctate’ or ‘granular’ distributions of ferritin that we observe (FIG. 5) and both are compatible with the non-random association of ferritin with different nuclear fractions (FIG. 6A). This pattern may extend to all other cell types.

Further investigations will examine the role of post-translational modification in ferritin translocation and in its turnover. Similarly, we would like to know with what molecular systems does ferritin associate during the translocation process and how is the process regulated. Finally, the fact that ferritin appears in the nucleus in response to environmental signals suggests that it plays a functional role in that compartment. The increasing content of ferritin in the soluble chromatin when compared with the nuclear matrix or nucleolar fractions leads us to speculate that ferritin may specifically bind some component of heterochromatin. If this is the case, we anticipate a functional role for ferritin, perhaps in iron sequestration or delivery, in the molecular transactions of chromatin.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention.

### Example 4

### In Vivo Tumor Inhibition

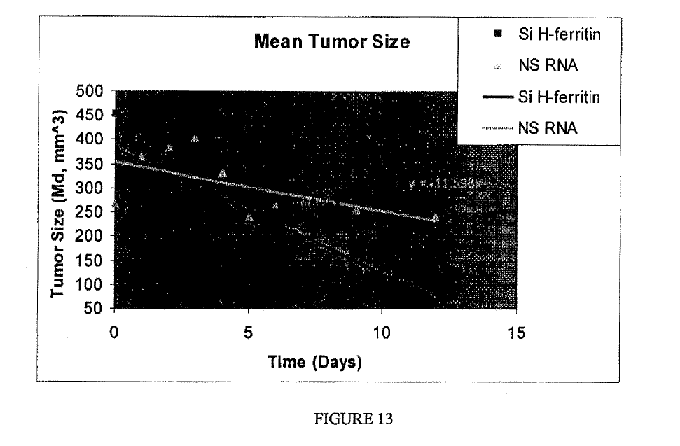

For this study, a subcutaneous tumor model was used to show the in vivo efficacy of the siRNA H-ferritin approach. The siRNA for H-ferritin or the nonsense (NS) control was first conjugated into liposomes and then injected directly into a subcutaneous glioblastoma tumor growing in the flank of nude mice. The concentration of siRNA or NS RNA injected into the tumor was ˜4 μg. After injection of the siRNA, the mice, received 25 μM of BCNU delivered i.p. 24 hours. The injections were performed once a week. As can be seen FIG. 13, the rate of tumor shrinkage was significantly faster in the animals receiving siRNA in the tumors as opposed to NS RNA. The significance of the data in this graph are two-fold: 1) the data provide proof of concept that siRNA for H-ferritin delivered into tumors enhance the efficacy of standard chemotherapeutic agents, 2) the siRNA can be delivered to the tumors using a liposome delivery system.

### Example 5

### Protective Effects of H-Ferritin

H-ferritin immunostaining in primary rat astrocytes: In normal astrocytes the immunostaining is almost undetectable. In contrast, H-ferritin i immunostaining is apparent in cell nuclei following transfection with a NLS containing c-myc H-ferritin construct. The nuclear staining is punctate and similar to that seen in non˜transfected cells that have nuclear ferritin. Cytoplasmic immunostaining for H-ferritin is also present with this system because ferritin is synthesized on the polysomes. The data show that at three days, is the first time that we can clearly detect the presence of the reaction product for ferritin in the nucleus. The non-NLS containing H-ferritin is only found in the cytoplasm at three days after transfection

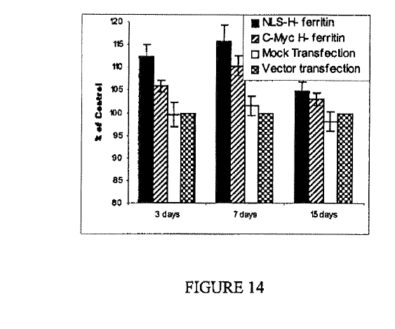

The effect of nuclear ferritin on cell viability: Transfected primary astrocytes were monitored for their growth rate. The results are shown in FIG. 14. The controls for these studies were cells transfected with vector alone and mock transfected cells (using H₂O). The experimental group consisted of transfection with a Nuclear localization signal on H-ferritin. For these studies cells were plated at equal density in 24 well plates after transfection. The transfected cells had normal morphology. The NLS-ferritin containing cells grow faster than the controls for the first 15 days. After 15 days the NLS-ferritin transfected astrocytes become confluent and stopped proliferating and the controls attain an equal density. This indicates that the transfected cells are not immortalized and maintain contact inhibition (i.e. are not cancerous).

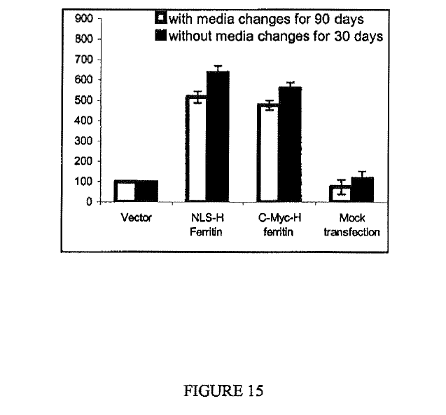

FIG. 15 shows the results of a second set of experiments that examined the longevity of transfected astrocytes versus control. Cells were transfected and then plated at equal density. The longevity experiments were performed in two ways. First, cell viability was monitored in cultures in which the media was changed every seven days. In the second experiment, the media was not changed for 30 days to test nutrient deprivation. An MTT assay was performed on randomly selected cell cultures from 24 well plates every two weeks to monitor cell viability. The results show that the cells transfected with ferritin survive for three months when the media was being changed or 30 days when the media was not changed. The non-transfected astrocytes, however, die at an increased rate compared to the ferritin transfected cells. We have continued the study out to five months on the transfected cells, but none of the non-transfected cells are alive. The transfected cells survive for the longer periods but there number does not change during this time.

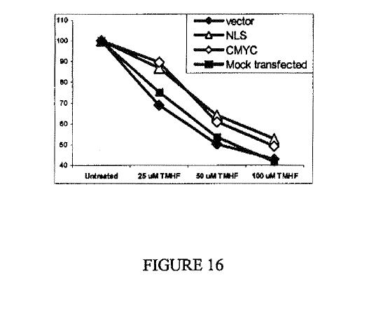

The studies shown in FIGS. 15 and 16 demonstrate three remarkable and novel findings. First, the cells transfected with NLS-ferritin construct showed increased survival and growth compared to controls, which is consistent with our hypothesis. Secondly, survival and growth of the cells transfected with non-NLS containing ferritin were consistently less than the cells transfected with NLS containing ferritin. Thirdly, the non-NLS ferritin translocated to the nucleus over time. Thus, the increase in survival of the astrocytes transfected with the non-NLS containing ferritin construct do not negate our line of reasoning and could be consistent with our claim that the effects we are seeing are due to nuclear ferritin.

The positive impact of nuclear ferritin on cell survival suggests that it will also protect these cells from cell stressors. The results of our initial studies are shown in FIG. 16.

FIG. 16 shows the cytotoxicity profile of stress factors on rat primarily astrocytes transfected with our NLS and non-NLS H ferritin construct. Cytotoxicity was determined by MTT assay after treatment with three different concentrations of soluble iron compound 3,5,5-trimethyl (hexanoyl) ferrocene (TMHF). A total of 12 trials were run in triplicate for each sample and the grand mean and SE are reported. These results demonstrate that transfection with ferritin increases the ability of primary astrocytes to withstand iron induced stress. Other common stressors (hydrogen peroxide, cytokines, hypoxia) will also be examined.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents. All references cited herein, are incorporated herein by reference.

1: A method of inhibiting cancerous tumor growth in a mammal comprising:
providing a composition comprising liposomes associated with an effective amount of siRNA directed against H-ferritin for decreasing H-ferritin expression in cancerous cells within 48 hours of administration, increasing sensitivity of cancerous cells to a chemotherapeutic drug, and inhibiting tumor growth in the mammal; and
administering the composition to a mammal having a cancerous tumor resulting in inhibition of growth of the cancerous tumor.

2: The method of claim 1, further comprising administering a chemotherapeutic drug to the mammal subsequent to administration of the composition to the mammal.

3: The method of claim 1, wherein administration of the composition comprises injection of the composition into the cancerous tumor.

4: The method of claim 1, wherein administration of the composition to the mammal increases sensitivity of cancerous cells to the chemotherapeutic drug.

5: The method of claim 1, wherein the cancerous tumor is a glioblastoma multiforme.

6: The method of claim 1, wherein the siRNA comprises the sequences of SEQ ID NOs: 1 and 2.

7: The method of claim 1, wherein the siRNA comprises the sequences of SEQ ID NOs: 3 and 4.

8: The method of claim 1, wherein the siRNA comprises the sequences of SEQ ID NOs: 5 and 6.

9: The method of claim 1, wherein the siRNA comprises the sequences of SEQ ID NOs: 7 and 8.

10: The method of claim 1, wherein the composition further comprises a pharmaceutically acceptable carrier.

11: The method of claim 1, wherein the liposomes comprise a tumor cell targeting moiety selected from the group consisting of: antibody, nucleic acid, and receptor ligand.

12: The method of claim 1, wherein the chemotherapeutic drug is associated with a particulate delivery vehicle.

13: The method of claim 1, wherein the chemotherapeutic drug is BCNU.

14: A method of inhibiting cancerous tumor growth in a mammal comprising:
providing a composition comprising liposomes associated with an effective amount of siRNA directed against H-ferritin for decreasing H-ferritin expression in cancerous cells within 48 hours of administration, increasing sensitivity of cancerous cells to radiation, and inhibiting tumor growth in the mammal; and
administering the composition to a mammal having a cancerous tumor resulting in inhibition of growth of the cancerous tumor and increased sensitivity of cancerous cells to radiation.

15: The method of claim 14, further comprising administering radiation to the mammal subsequent to administration of the composition to the mammal.

16: The method of claim 14, wherein the liposomes are cationic liposomes.

17: The method of claim 14, wherein the cancerous tumor is a glioblastoma multiforme.

18: The method of claim 14, wherein the siRNA comprises the sequences of SEQ ID NOs: 1 and 2, or SEQ ID NOs: 3 and 4, or SEQ ID NOs: 5 and 6, or SEQ ID NOs: 7 and 8.

19: The method of claim 1, wherein the composition further comprises a pharmaceutically acceptable carrier.

20: The method of claim 1, wherein the liposomes comprise a tumor cell targeting moiety selected from the group consisting of: antibody, nucleic acid, and receptor ligand.


SEQUENCE LISTING (10 sequences)

SEQ ID NO: 1 - RNA, 20 nt, Artificial Sequence
  chemically synthesized
gccagaacuaccaccaggac

SEQ ID NO: 2 - RNA, 20 nt, Artificial Sequence
  Chemically Synthesized
cggucuugauggugguccug

SEQ ID NO: 3 - RNA, 20 nt, Artificial Sequence
  Chemically Synthesized
guggcuuugaagaacuuugc

SEQ ID NO: 4 - RNA, 20 nt, Artificial Sequence
  Chemically Synthesized
caccgaaacuucuugaaacg

SEQ ID NO: 5 - RNA, 20 nt, Artificial Sequence
  Chemically Synthesized
gaaucagucacuacuggaac

SEQ ID NO: 6 - RNA, 20 nt, Artificial Sequence
  Chemically Synthesized
cuuagucugugaugaccuug

SEQ ID NO: 7 - RNA, 20 nt, Artificial Sequence
  Chemically Synthesized
ggaauaucucuuugacaagc

SEQ ID NO: 8 - RNA, 20 nt, Artificial Sequence
  Chemically Synthesized
ccuuauagagaaacuguucg

SEQ ID NO: 9 - protein, 182 aa, mammalian
TTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 10 - protein, 174 aa, mammalian
SSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD